SEQUENCE LISTING (70 sequences)

SEQ ID NO: 1 - protein, 5 aa, synthetic construct
  REGION 1..5: note = VH CDR 1 (construct 3)
SYWIG

SEQ ID NO: 2 - protein, 17 aa, synthetic construct
  REGION 1..17: note = VH CDR2 (construct 3)
IIYPGDSDTRYSPSFQG

SEQ ID NO: 3 - protein, 13 aa, synthetic construct
  REGION 1..13: note = VH CDR3 (construct 3)
QAIAVTGLGGFDP

SEQ ID NO: 4 - protein, 12 aa, synthetic construct
  REGION 1..12: note = VL CDR1 (construct 3)
RASQSVSSSYLA

SEQ ID NO: 5 - protein, 7 aa, synthetic construct
  REGION 1..7: note = VL CDR2 (construct 3)
GASSRAT

SEQ ID NO: 6 - protein, 9 aa, synthetic construct
  REGION 1..9: note = VL CDR3 (construct 3)
QQYDTSPFT

SEQ ID NO: 7 - protein, 7 aa, synthetic construct
  REGION 1..7: note = VH CDR1 (construct 7)
SSSAAWN

SEQ ID NO: 8 - protein, 18 aa, synthetic construct
  REGION 1..18: note = VH CDR2 (construct 7)
HIGYRSKWYNEYAVSVKS

SEQ ID NO: 9 - protein, 16 aa, synthetic construct
  REGION 1..16: note = VH CDR3 (construct 7)
GMYGSVPYKEGYYFDI

SEQ ID NO: 10 - protein, 11 aa, synthetic construct
  REGION 1..11: note = VL CDR1 (construct 7)
RASQGISSDLN

SEQ ID NO: 11 - protein, 7 aa, synthetic construct
  REGION 1..7: note = VL CDR2 (construct 7)
AASNLQS

SEQ ID NO: 12 - protein, 9 aa, synthetic construct
  REGION 1..9: note = VL CDR3 (construct 7)
QQYTDESMT

SEQ ID NO: 13 - protein, 5 aa, synthetic construct
  REGION 1..5: note = VH CDR1 (construct 8)
GYHMS

SEQ ID NO: 14 - protein, 16 aa, synthetic construct
  REGION 1..16: note = VH CDR2 (construct 8)
VINPVSGNTVYAQKFQ

SEQ ID NO: 15 - protein, 10 aa, synthetic construct
  REGION 1..10: note = VH CDR3 (construct 8)
IPSYTYAFDY

SEQ ID NO: 16 - protein, 11 aa, synthetic construct
  REGION 1..11: note = VL CDR1 (construct 8)
RASQDISNYLA

SEQ ID NO: 17 - protein, 7 aa, synthetic construct
  REGION 1..7: note = VL CDR2 (construct 8)
RASSLQS

SEQ ID NO: 18 - protein, 9 aa, synthetic construct
  REGION 1..9: note = VL CDR3 (construct 8)
QQHGHSPTT

SEQ ID NO: 19 - protein, 9 aa, synthetic construct
  REGION 1..9: note = VH CDR1 (construct 10)
FTFSNYYMA

SEQ ID NO: 20 - protein, 10 aa, synthetic construct
  REGION 1..10: note = VH CDR2 (construct 10)
SLTNSGGSTY

SEQ ID NO: 21 - protein, 7 aa, synthetic construct
  REGION 1..7: note = VH CDR3 (construct 10)
EWAGSGY

SEQ ID NO: 22 - protein, 7 aa, synthetic construct
  REGION 1..7: note = VL CDR1 (construct 10)
NVGNNLA

SEQ ID NO: 23 - protein, 7 aa, synthetic construct
  REGION 1..7: note = VL CDR2 (construct 10)
YTSNRFT

SEQ ID NO: 24 - protein, 8 aa, synthetic construct
  REGION 1..8: note = VL CDR3 (construct 10)
RIYNSPWT

SEQ ID NO: 25 - protein, 120 aa, synthetic construct
  REGION 1..120: note = VH chain (construct 3)
EVQLVQSGAEVKKPGESLKISCKGSGYTFTSYWIGWVRQMPGKGLEWMGIIYPGDSDTRYSPSFQGQVTISVDKSSSTAYLQWSSLKASDTAIYYCTRQAIAVTGLGGFDPWGQGTLVTV

SEQ ID NO: 26 - protein, 108 aa, synthetic construct
  REGION 1..108: note = VL chain (construct 3)
EIVLTQSPGTLSLSPGERATLSCRASQSVSSSYLAWYQQKPGQAPRLLIYGASSRATGIPDRFSGSGSGTDFTLTISRLEPEDFALYYCQQYDTSPFTFGPGTKVDIK

SEQ ID NO: 27 - protein, 126 aa, synthetic construct
  REGION 1..126: note = VH chain (construct 7)
QVQLQQSGPGLVKPSQTLSLTCAISGDSVSSSSAAWNWIRQSPSRGLEWLGHIGYRSKWYNEYAVSVKSRITINPDTSKNQFSLQLNSVTPEDTAVYYCARGMYGSVPYKEGYYFDIWGQGTLVTV

SEQ ID NO: 28 - protein, 107 aa, synthetic construct
  REGION 1..107: note = VL chain (construct 7)
DIQMTQSPSSLSASVGDRVTITCRASQGISSDLNWYQQKPGKAPKLLIYAASNLQSGVPSRFSGSGSGTDFTLTISSLQPEDFATYYCQQYTDESMTFGQGTKVEIK

SEQ ID NO: 29 - protein, 117 aa, synthetic construct
  REGION 1..117: note = VH chain (construct 8)
QVQLVQSGAEVKKPGASVKVSCKASGYTFTGYHMSWVRQAPGQGLEWMGVINPVSGNTVYAQKFQGRVTMTRDTSISTAYMELSRLRSEDTAVYYCARIPSYTYAFDYWGQGTLVTV

SEQ ID NO: 30 - protein, 107 aa, synthetic construct
  REGION 1..107: note = VL chain (construct 8)
DIQMTQSPSSLSASVGDRVTITCRASQDISNYLAWYQQKPGKAPKLLIYRASSLQSGVPSRFSGSGSGTDFTLTISSLQPEDFATYYCQQHGHSPTTFGQGTKVEIK

SEQ ID NO: 31 - protein, 117 aa, synthetic construct
  REGION 1..117: note = VH chain (construct 10)
EVQLLESGGGLVQPGGSLRLSCAASGFTFSNYYMAWVRQAPGKGLEWVSSLTNSGGSTYYADSVKGRFTISRDNSKNTLYLQMNSLRAEDTAVYYCTREWAGSGYFDYWGQGTLVTV

SEQ ID NO: 32 - protein, 107 aa, synthetic construct
  REGION 1..107: note = VL chain (construct 10)
DIQMTQSPSSLSASVGDRVTMTCKASQNVGNNLAWYQQKPGKAPKLLLYYTSNRFTGVPSRFSGSGSGTDFTLTISSVQPEDFATYYCQRIYNSPWTFGQGTKLELK

SEQ ID NO: 33 - protein, 245 aa, synthetic construct
  REGION 1..245: note = scFv (construct 3)
EVQLVQSGAEVKKPGESLKISCKGSGYTFTSYWIGWVRQMPGKGLEWMGIIYPGDSDTRYSPSFQGQVTISVDKSSSTAYLQWSSLKASDTAIYYCTRQAIAVTGLGGFDPWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFALYYCQQYDTSPFTFGPGTKVDIK

SEQ ID NO: 34 - protein, 250 aa, synthetic construct
  REGION 1..250: note = scFv (construct 7)
QVQLQQSGPGLVKPSQTLSLTCAISGDSVSSSSAAWNWIRQSPSRGLEWLGHIGYRSKWYNEYAVSVKSRITINPDTSKNQFSLQLNSVTPEDTAVYYCARGMYGSVPYKEGYYFDIWGQGTLVTVSSGGGGSGGGGSGGGGSDIQMTQSPSSLSASVGDRVTITCRASQGISSDLNWYQQKPGKAPKLLIYAASNLQSGVPSRFSGSGSGTDFTLTISSLQPEDFATYYCQQYTDESMTFGQGTKVEIK

SEQ ID NO: 35 - protein, 241 aa, synthetic construct
  REGION 1..241: note = scFv (construct 8)
QVQLVQSGAEVKKPGASVKVSCKASGYTFTGYHMSWVRQAPGQGLEWMGVINPVSGNTVYAQKFQGRVTMTRDTSISTAYMELSRLRSEDTAVYYCARIPSYTYAFDYWGQGTLVTVSSGGGGSGGGGSGGGGSDIQMTQSPSSLSASVGDRVTITCRASQDISNYLAWYQQKPGKAPKLLIYRASSLQSGVPSRFSGSGSGTDFTLTISSLQPEDFATYYCQQHGHSPTTFGQGTKVEIK

SEQ ID NO: 36 - protein, 241 aa, synthetic construct
  REGION 1..241: note = scFv (construct 10)
EVQLLESGGGLVQPGGSLRLSCAASGFTFSNYYMAWVRQAPGKGLEWVSSLTNSGGSTYYADSVKGRFTISRDNSKNTLYLQMNSLRAEDTAVYYCTREWAGSGYFDYWGQGTLVTVSSGGGGSGGGGSGGGGSDIQMTQSPSSLSASVGDRVTMTCKASQNVGNNLAWYQQKPGKAPKLLLYYTSNRFTGVPSRFSGSGSGTDFTLTISSVQPEDFATYYCQRIYNSPWTFGQGTKLELK

SEQ ID NO: 37 - protein, 17 aa, synthetic construct
  REGION 1..17: note = linker sequence
SSGGGGSGGGGSGGGGS

SEQ ID NO: 38 - protein, 516 aa, synthetic construct
  REGION 1..516: note = construct 3
MASPLTRFLSLNLLLLGESIILGSGEAEVQLVQSGAEVKKPGESLKISCKGSGYTFTSYWIGWVRQMPGKGLEWMGIIYPGDSDTRYSPSFQGQVTISVDKSSSTAYLQWSSLKASDTAIYYCTRQAIAVTGLGGFDPWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFALYYCQQYDTSPFTFGPGTKVDIKLRVFSSVVPVLQKVNSTTTKPVLRTPSPVHPTGTSQPQRPEDCRPRGSVKGTGLDFACDIYIWAPLAGICVALLLSLIITLICYHRQLWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 39 - protein, 521 aa, synthetic construct
  REGION 1..521: note = construct 7
MASPLTRFLSLNLLLLGESIILGSGEAQVQLQQSGPGLVKPSQTLSLTCAISGDSVSSSSAAWNWIRQSPSRGLEWLGHIGYRSKWYNEYAVSVKSRITINPDTSKNQFSLQLNSVTPEDTAVYYCARGMYGSVPYKEGYYFDIWGQGTLVTVSSGGGGSGGGGSGGGGSDIQMTQSPSSLSASVGDRVTITCRASQGISSDLNWYQQKPGKAPKLLIYAASNLQSGVPSRFSGSGSGTDFTLTISSLQPEDFATYYCQQYTDESMTFGQGTKVEIKLRVFSSVVPVLQKVNSTTTKPVLRTPSPVHPTGTSQPQRPEDCRPRGSVKGTGLDFACDIYIWAPLAGICVALLLSLIITLICYHRQLWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 40 - protein, 512 aa, synthetic construct
  REGION 1..512: note = construct 8
MASPLTRFLSLNLLLLGESIILGSGEAQVQLVQSGAEVKKPGASVKVSCKASGYTFTGYHMSWVRQAPGQGLEWMGVINPVSGNTVYAQKFQGRVTMTRDTSISTAYMELSRLRSEDTAVYYCARIPSYTYAFDYWGQGTLVTVSSGGGGSGGGGSGGGGSDIQMTQSPSSLSASVGDRVTITCRASQDISNYLAWYQQKPGKAPKLLIYRASSLQSGVPSRFSGSGSGTDFTLTISSLQPEDFATYYCQQHGHSPTTFGQGTKVEIKLRVFSSVVPVLQKVNSTTTKPVLRTPSPVHPTGTSQPQRPEDCRPRGSVKGTGLDFACDIYIWAPLAGICVALLLSLIITLICYHRQLWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 41 - protein, 512 aa, synthetic construct
  REGION 1..512: note = construct 10
MASPLTRFLSLNLLLLGESIILGSGEAEVQLLESGGGLVQPGGSLRLSCAASGFTFSNYYMAWVRQAPGKGLEWVSSLTNSGGSTYYADSVKGRFTISRDNSKNTLYLQMNSLRAEDTAVYYCTREWAGSGYFDYWGQGTLVTVSSGGGGSGGGGSGGGGSDIQMTQSPSSLSASVGDRVTMTCKASQNVGNNLAWYQQKPGKAPKLLLYYTSNRFTGVPSRFSGSGSGTDFTLTISSVQPEDFATYYCQRIYNSPWTFGQGTKLELKLRVFSSVVPVLQKVNSTTTKPVLRTPSPVHPTGTSQPQRPEDCRPRGSVKGTGLDFACDIYIWAPLAGICVALLLSLIITLICYHRQLWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 42 - protein, 27 aa, synthetic construct
  REGION 1..27: note = CD8a leader/signal peptide (murine)
MASPLTRFLSLNLLLLGESIILGSGEA

SEQ ID NO: 43 - protein, 83 aa, synthetic construct
  REGION 1..83: note = CD8a hinge and transmembrane domain (murine)
FSSVVPVLQKVNSTTTKPVLRTPSPVHPTGTSQPQRPEDCRPRGSVKGTGLDFACDIYIWAPLAGICVALLLSLIITLICYHR

SEQ ID NO: 44 - protein, 43 aa, synthetic construct
  REGION 1..43: note = CD28 signalling domain (murine)
WTNSRRNRLLQSDYMNMTPRRPGLTRKPYQPYAPARDFAAYRP

SEQ ID NO: 45 - protein, 113 aa, synthetic construct
  REGION 1..113: note = CD3z endodomain (murine)
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 46 - protein, 19 aa, synthetic construct
  REGION 1..19: note = P2a peptide cleavage domain
ATNFSLLKQAGDVEENPGP

SEQ ID NO: 47 - protein, 18 aa, synthetic construct
  REGION 1..18: note = T2A peptide cleavage domain
EGRGSLLTCGDVEENPGP

SEQ ID NO: 48 - protein, 20 aa, synthetic construct
  REGION 1..20: note = E2A peptide cleavage domain
QCTNYALLKLAGDVESNPGP

SEQ ID NO: 49 - protein, 22 aa, synthetic construct
  REGION 1..22: note = F2A peptide cleavage domain
VKQTLNFDLLKLAGDVESNPGP

SEQ ID NO: 50 - protein, 42 aa, synthetic construct
  REGION 1..42: note = OX40 signalling domain
ALYLLRRDQRLPPDAHKPPGGGSFRTPIQEEQADAHSTLAKI

SEQ ID NO: 51 - protein, 42 aa, synthetic construct
  REGION 1..42: note = 41BB signalling domain
KRGRKKLLYIFKQPFMRPVQTTQEEDGCSCRFPEEEEGGCEL

SEQ ID NO: 52 - protein, 38 aa, synthetic construct
  REGION 1..38: note = ICOS signalling domain
CWLTKKKYSSSVHDPNGEYMFMRAVNTAKKSRLTDVTL

SEQ ID NO: 53 - protein, 197 aa, synthetic construct
  REGION 1..197: note = TNFRSF25 signalling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 54 - protein, 286 aa, synthetic construct
  REGION 1..286: note = IL2RB
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 55 - protein, 94 aa, synthetic construct
  REGION 1..94: note = IL2RB truncated - Y510
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 56 - protein, 208 aa, synthetic construct
  REGION 1..208: note = IL2RB truncated - Y510 & Y392
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 57 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 58 - protein, 431 aa, synthetic construct
  REGION 1..431: note = FOXP3, aa418 mutant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPSRCSNPTPGP

SEQ ID NO: 59 - protein, 431 aa, synthetic construct
  REGION 1..431: note = FOXP3, aa422 mutant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPARCSNPTPGP

SEQ ID NO: 60 - protein, 431 aa, synthetic construct
  REGION 1..431: note = FOXP3, aa 418 and 422 mutant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPARCSNPTPGP

SEQ ID NO: 61 - protein, 362 aa, synthetic construct
  REGION 1..362: note = FOXP3, truncated variant
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 62 - protein, 441 aa, synthetic construct
  REGION 1..441: note = FOXP3, illustrative variant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 63 - DNA, 1296 bp, synthetic construct
  misc_feature 1..1296: note = FOXP3, Illustrative FOXP3 polynucleotide
  source 1..1296: mol_type = other DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 64 - DNA, 1352 bp, synthetic construct
  misc_feature 1..1352: note = FOXP3, Illustrative FOXP3 polynucleotide
  source 1..1352: mol_type = other DNA
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 65 - skipped

SEQ ID NO: 66 - protein, 4 aa, synthetic construct
  REGION 1..4: note = Furin cleavage site
RRKR

SEQ ID NO: 67 - DNA, 406 bp, synthetic construct
  misc_feature 1..406: note = SFFV promoter
  source 1..406: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcggccg

SEQ ID NO: 68 - protein, 230 aa, Homo sapiens
  REGION 1..230: note = TREM2 human isoform 1
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 69 - protein, 174 aa, synthetic construct
  REGION 1..174: note = TREM2 exodomain
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTS

SEQ ID NO: 70 - protein, 156 aa, synthetic construct [sequence omitted]

(19) United States
(12) Patent Application Publication
(10) Pub. No.: US 2024/0390422 A1
(43) Pub. Date: Nov. 28, 2024

(54) ANTI-TREM2 CHIMERIC ANTIGEN RECEPTOR

(71) Applicant: QUELL THERAPEUTICS LIMITED, London (GB)

(72) Inventors: Iain MCGILL, London (GB); Luke HENRY, London (GB)

(21) Appl. No.: 18/694,341

(22) PCT Filed: Sep. 21, 2022
(86) PCT No.: PCT/GB2022/052385

§ 371 Date: Mar. 21, 2024

(30) Foreign Application Priority Data
Sep. 21, 2021 (GB) 2113449.9
Dec. 16, 2021 (GB) 2118273.8
May 11, 2022 (GB) 2206884.5

(51) Int. Cl. A61K 35/17; A61K 39/00; A61P 25/00; A61P 29/00; C07K 16/28; C12N 5/0783

(52) U.S. Cl. CPC A61K 35/17; A61K 39/4611; A61K 39/4631; A61K 39/464411; A61P 25/00; A61P 29/00; C07K 16/2803; C12N 5/0637; A61K 2239/13; A61K 2239/17; A61K 2239/21; A61K 2239/22

(57) ABSTRACT

The present invention relates to chimeric antigen receptors (CARs), particularly CARs expressed in immune cells (e.g. Tregs) and their use in therapy. In particular, the invention provides a CAR comprising an antigen recognition domain that specifically binds to TREM2.

### FIELD OF THE INVENTION

The present invention relates generally to the field of chimeric antigen receptors, TREM2 biology and related therapies, such as the treatment of neurological disorders characterized by neuronal damage, neuroinflammation or neurodegeneration. More particularly, the invention provides chimeric antigen receptors (CARs) that bind to TREM2 and that are expressed in immune cells (e.g., Tregs). Such immune cells have therapeutic uses in diseases and conditions associated with the accumulation of TREM2-expressing cells and/or soluble TREM2. The invention further provides nucleic acid molecules encoding such CARs and vectors containing them that may be used to modify host cells, e.g., immune cells, to express the CARs.

### BACKGROUND TO THE INVENTION

Inflammation is the body's biological response to injury and infection and functions to eliminate the initial cause of cell injury and effect repair. However, an immune response that results in chronic inflammation can lead to tissue damage and ultimately its destruction. Chronic inflammation is often a result of an inappropriate immune response.

For many diseases associated with inflammation, such as neurodegeneration, there are currently few treatments and prognosis is poor. For instance, Amyotrophic lateral sclerosis (ALS), also known as Motor Neuron Disease or Lou Gehrig's disease, is a devastating neurodegenerative disease which is characterised by the degeneration of both the upper (neurons projecting from cortex to brainstem) and lower (neurons projecting from the brainstem or spinal cord to muscle) motor neurones, resulting in muscular weakness and eventual respiratory failure.

ALS is defined as an ‘orphan disease’ with an incidence of approximately 1.5-5 per 100,000 people per year in North America and Europe. The median survival is 2-5 years from symptom onset with death typically resulting from respiratory failure. The mean age of onset is 56 though ALS can affect people of any age and is more frequent in men, with a male to female ratio of approximately 3 to 2.

The current standard of care for ALS patients is a multi-disciplinary approach to manage symptoms and increase quality of life through pharmacological and non-pharmacological interventions. Although over fifty drugs with different mechanisms of action have been assessed for the treatment of ALS, only two compounds, Riluzole and Edaravone, have come to market in the US with very modest efficacy (Riluzole increased survival by approximately 3 months after 18 months of treatment, compared with placebo (Bensimon et al., 1994, N Engl J Med, 330:585-591)), and only Riluzole has been approved for use in the UK, leaving considerable unmet need.

The story is similar for other neurodegenerative diseases associated with inflammation, such as frontotemporal dementia (FTD), progressive supranuclear palsy (PSP), Parkinson's disease, Alzheimer's disease, Huntington's Disease and Multiple Sclerosis, and there is a significant need for products that can slow progression of these diseases and extend survival.

Although the aetiology of these diseases is not fully understood, inflammation in the nervous system (“neuroinflammation”) is a common pathological hallmark of disease and can be particularly harmful, especially when sustained for a long period. Even though inflammation may not be disease-causing in and of itself, it can contribute to disease pathogenesis across both the peripheral nervous system (e.g., neuropathic pain, fibromyalgia) and the central nervous system (e.g., amyotrophic lateral sclerosis (ALS), Alzheimer disease, Parkinson's disease, multiple sclerosis and other demyelinating diseases, ischemic and traumatic brain injury, depression, and autism spectrum disorder). Communication between the nervous system and immune system may represent an important factor in neuroinflammation.

In ALS, for example, the most important clinical finding at the site of neuronal damage is neuroinflammation, which is caused by the activation of microglia (resident macrophages within the CNS), astrocytes, and infiltration of monocytes and T cells (Zhao, Beers, & Appel, 2013, Journal of Neuroimmune Pharmacology, 8(4), 888-899). This has been confirmed by examination of ALS patient samples (spinal cords, brains, CSF samples) as well as use of transgenic mouse models of disease, the most prominent of which is the mSOD1 model.

Microglia are macrophage-like myeloid cells that play a critical role in maintaining the homeostasis of the nervous system by regulating cell death and neurogenesis and by contributing to synaptic pruning during postnatal development. A key property of microglia is their capacity to modify their activation status based on environmental changes. Thus, upon neuronal injury or in neurodegenerative/neuroinflammatory diseases, microglia become activated and exert protective effects such as phagocytosis of cell debris and secretion of neurotrophic mediators. However, under some circumstances, microglia amplify the damage of the nervous tissue by producing toxic molecules, releasing cytokines/chemokines, presenting antigens to T cells and phagocytosing the injured neurones. Considering that the proportion of the global population suffering from neuroinflammatory and neurodegenerative disorders is increasing, there is an urgent need for new therapies to modulate microglial activity and plasticity as a means to treat these diseases.

TREM2 is a type 1 transmembrane protein member of the Ig superfamily that is expressed on microglia (and other myeloid cell subsets), and binds anionic lipids and DNA released during neuronal and glial damage, as well as other molecules such as amyloid beta oligomers (AR). TREM2 forms a complex with DAP12, which, upon ligand binding to TREM2, transduces signals into the cytoplasm through its ITAM motifs. Whilst TREM2 has been reported to support microglial metabolism and to promote the migration, cytokine release, phagocytosis, proliferation and survival of the cells, the role of TREM2 within disease conditions is complicated. Particularly, the role of TREM2 as a pro or anti-inflammatory molecule is unclear.

CD4+Foxp3+ regulatory T cells (Tregs) are a lymphocyte subset that is essential for the maintenance of dominant immunological tolerance by inhibiting the function of various effector immune cell subsets, including myeloid cells such as macrophages and dendritic cells. In addition, Tregs are also known to promote tissue repair and regeneration. Tregs are known to be involved in controlling neuroinflammatory disorders such as multiple sclerosis, amyotrophic lateral sclerosis, as well as ischemic and traumatic brain injury.

In ALS, for example, Tregs are associated with progression rates of the disease. In a normal CNS, resting microglia and Tregs provide immune surveillance of the neuronal environment. In the early stages of ALS, microglia with an M2 phenotype have a neuroprotective effect. However, as the disease advances, neurons release misfolded proteins (which are often misfolded due to genetic mutations in the coding of the protein) including mutant SOD1, mutant FUS, mutant TDP-43 and dipeptide repeat proteins (DPRs) derived from expanded C9orf72 (Ferrara et al., 2018, Front Neurosci, 12: 574). These proteins induce the activation of microglia with an M1 phenotype. M1 microglia produce pro-inflammatory cytokines, release reactive oxygen species (ROS) and activate astrocytes, which do the same. In the early stages of disease, Tregs can polarize these M1 microglia back to a protective M2 phenotype and they can also suppress the induction of T effector cells (Teffs). However, in later stages, immune responses from Tregs shift to T helper (Th)1/Th17 cells and the M1 microglia are no longer polarized to an M2 phenotype (Machhi et al., 2020, Mol. Neurodegener., 15:32). This ultimately causes more inflammation and damage to the neurons (Zhao, Beers, & Appel, 2013, Journal of Neuroimmune Pharmacology, 8(4), 888-899). Furthermore, as ALS progresses, Treg numbers are significantly decreased and the cells gradually lose their regulatory functions (Henkel et al., 2013, EMBO Molecular Medicine, 5(1), 64-79; Beers et al., 2017, JCI Insight, 2(5); Sheean et al., 2018, JAMA Neurology, 75(6), 681-689).

Non-specific polyclonal Tregs have been shown to be useful in the treatment of autoimmune, inflammatory, and neurodegenerative diseases, including in ALS (Thonhoff et al., 2018, Neurol Neuroimmunol Neuroinflamm, 18; 5(4):e465) but these polyclonal cells may be associated with unwanted effects, such as systemic immunosuppression

The prospect of ameliorating immunopathology and re-establishing tolerance in inflammatory diseases has prompted a growing interest in the clinical development of Treg-based immunotherapies. For a Treg immunotherapy to be successful it is essential to develop strategies that promote the trafficking of Tregs to the site of tissue damage and induce their activation in situ.

### SUMMARY OF THE INVENTION

The present inventors have determined that a generic therapy for the treatment of neurodegenerative and other conditions associated with the TREM2 pathway (i.e., associated with the accumulation of soluble TREM2 or TREM2-expressing cells) may be developed by providing immune cell subsets with a chimeric antigen receptor comprising an antigen recognition domain that is specific for TREM2.

In particular, the inventors have found that the CAR-Tregs specific for TREM2 are activated in the presence of antigen.

The expression of such a CAR on the surface of Tregs provides a generic therapy that can be used for the treatment of conditions associated with inflammation, where TREM2 is expressed locally at the site of disease, in view of the well-known bystander effect of Tregs and their ability, once activated, to reduce the immune response and modulate the activation status of myeloid cells and other immune cell subsets.

In ALS, for example, CAR-Tregs specific for TREM2 may traffic to inflammatory lesions surrounding the upper and lower motor neurons in the CNS where they may modulate the local immune environment, in particular microglia phenotype.

Accordingly, in one aspect, the present invention provides a chimeric antigen receptor (CAR) comprising an antigen recognition domain that specifically binds to TREM2 (e.g., to human TREM2).

In this respect, there is provided herein a CAR comprising:
a. an exodomain comprising the antigen recognition domain;
b. a transmembrane domain; and
c. an endodomain comprising an intracellular signalling domain.

The CAR may further comprise a hinge domain and/or one or more co-stimulatory domains. The hinge domain may be selected from the hinge regions of CD28, CD8α, CD4, CD7, CH2CH3, an immunoglobulin, or a part or variant thereof. Preferably, the CAR may comprise a CD8α or CH2CH3 hinge domain. The co-stimulatory domain may be selected from the intracellular domains of CD28, ICOS, CD134 (OX40), CD137 (4-1BB), CD27, or TNFRSF25, or a part or variant thereof. Preferably, the CAR may comprise a CD28 co-stimulatory domain.

The CAR may comprise one or more transmembrane domains, which may be selected from the transmembrane domains of CD28, ICOS, CD8α, CD4, CD134 (OX40), CD137 (4-1BB), CD3 zeta, CD45, CD9, CD16, CD22, CD33, CD64, CD80, CD86, CD154, CH2CH3, or a part or variant thereof. Preferably, the CAR may comprise a CD8α or CH2CH3 transmembrane domain.

The CAR may comprise one or more intracellular signalling domains selected from the group consisting of the CD3 zeta signalling domain or any of its homologs, a CD3 polypeptide, a syk family tyrosine kinase, a src family tyrosine kinase, CD2, CD5 and CD8, or a part of variant thereof. Preferably, the CAR may comprise the CD3 zeta signalling domain.

In one embodiment, the CAR may comprise a CD8α or CH2CH3 hinge domain (i.e., a hinge domain derived from CD8α or CH2CH3); a CD8α or CH2CH3 transmembrane domain (i.e, a transmembrane domain derived from CD8α or CH2CH3); a CD28 co-stimulatory domain (i.e., a co-stimulatory domain derived from CD28; and the CD3 zeta signalling domain (i.e., a signalling domain derived from CD3 zeta), wherein when the hinge domain is CD8α, the transmembrane domain is CD8α and when the hinge domain is CH2CH3, the transmembrane domain is CH2CH3. Alternatively viewed, in one embodiment, the CAR may comprise a CD8α hinge domain, a CD8α transmembrane domain, a CD28 co-stimulatory domain, and the CD3 zeta signalling domain. In a separate embodiment, the CAR may comprise a CH2CH3 hinge domain, a CH2CH3 transmembrane domain, a CD28 co-stimulatory domain, and the CD3 zeta signalling domain. Further, the CAR may comprise a CD28 transmembrane domain (i.e., a transmembrane domain derived from CD28), particularly in combination with a CD28 co-stimulatory domain.

The CAR of the present invention may comprise a signal peptide and/or a reporter peptide. In one embodiment, the polynucleotide sequence encoding a CAR of the present invention may comprise a further polynucleotide sequence encoding a reporter peptide linked by a self-cleaving or cleavage domain.

Furthermore, the endodomain of the CAR may comprise a STAT5 association motif, a JAK1 and/or JAK 2 binding motif and optionally a JAK 3 binding motif, preferably wherein the endodomain of the CAR comprises one or more sequences from an endodomain of an interleukin receptor (IL) receptor.

The CAR of the invention may comprise:
(i) an antigen binding domain which comprises VH CDR1, 2 and 3 sequences as set forth in SEQ ID NOs: 1, 2 and 3 respectively and VL CDR1, 2 and 3 sequences as set forth in SEQ ID NOs: 4, 5 and 6 respectively, or
(ii) an antigen binding domain which comprises VH CDR1, 2 and 3 sequences as set forth in SEQ ID NOs: 7, 8 and 9 respectively and VL CDR1, 2 and 3 sequences as set forth in SEQ ID NOs: 10, 11 and 12 respectively, or
(iii) an antigen binding domain which comprises VH CDR1, 2 and 3 sequences as set forth in SEQ ID NOs: 13, 14 and 15 respectively and VL CDR1, 2 and 3 sequences as set forth in SEQ ID NOs: 16, 17 and 18 respectively, or
(iv) an antigen binding domain which comprises VH CDR1, 2 and 3 sequences as set forth in SEQ ID NOs: 19, 20 and 21 respectively and VL CDR1, 2 and 3 sequences as set forth in SEQ ID NOs: 22, 23 and 24 respectively,
   wherein one or more of said CDR sequences of (i)-(iv) may optionally comprise 1 to 3 amino acid modifications relative to an aforementioned CDR sequence, particularly wherein one or more of said CDR sequences may optionally be modified by substitution, addition or deletion of 1 to 3 amino acids.

Also in this respect, the antigen binding domain of the CAR may comprise:
(i) a VH domain comprising the sequence as set forth in SEQ ID NO: 25, or a sequence having at least 70% sequence identity thereto, and a VL domain comprising the sequence as set forth in SEQ ID NO: 26, or a sequence having at least 70% identity thereto; or
(ii) a VH domain comprising the sequence as set forth in SEQ ID NO: 27, or a sequence having at least 70% sequence identity thereto, and a VL domain comprising the sequence as set forth in SEQ ID NO: 28, or a sequence having at least 70% identity thereto; or
(iii) a VH domain comprising the sequence as set forth in SEQ ID NO: 29, or a sequence having at least 70% sequence identity thereto, and a VL domain comprising the sequence as set forth in SEQ ID NO: 30, or a sequence having at least 70% identity thereto; or
(iv) a VH domain comprising the sequence as set forth in SEQ ID NO: 31, or a sequence having at least 70% sequence identity thereto, and a VL domain comprising the sequence as set forth in SEQ ID NO: 32, or a sequence having at least 70% identity thereto.

Furthermore, the antigen binding domain of the CAR may comprise:
(i) The sequence as set forth in SEQ ID NO: 33 or a sequence having at least 80% identity thereto; or
(ii) The sequence as set forth in SEQ ID NO: 34 or a sequence having at least 80% identity thereto; or
(iii) The sequence as set forth in SEQ ID NO: 35 or a sequence having at least 80% identity thereto; or
(iv) The sequence as set forth in SEQ ID NO: 36 or a sequence having at least 80% identity thereto.

In a second aspect, the invention provides a nucleic acid molecule encoding a CAR according to the invention.

In a third aspect, the invention provides a vector comprising a nucleic acid according to the invention. The vector may further comprise a nucleic acid molecule encoding a FOXP3 polypeptide, or a derivative or variant thereof.

In a further aspect, the invention provides a cell comprising a CAR, nucleic acid molecule or vector according to the invention. The cell may be an immune cell or a progenitor or precursor thereof. Preferably, the cell may be a T cell, or a precursor thereof, or a stem cell. In particular, the cell may be a Treg, or a precursor thereof, or an iPSC cell. The cell may be provided in a cell population, which forms a further aspect of the invention. The cell may be a production host cell.

The invention also provides a pharmaceutical composition comprising a cell, cell population or vector according to the invention.

In another aspect, the invention provides a cell, cell population or pharmaceutical composition according to the invention for use in therapy.

Further, the invention provides a cell, cell population or pharmaceutical composition of the invention for use in treating and/or preventing a neurological disease, disorder or injury, such as a neurodegenerative disease, or autoimmune or inflammatory disease, or for use in inducing immunosuppression, or for use in promoting tissue repair and/or tissue regeneration. The therapy may be adoptive cell transfer therapy.

Alternatively viewed, the invention provides a method for treating and/or preventing a neurological disease, disorder or injury, such as a neurodegenerative disease, or autoimmune or inflammatory disease, or for inducing immunosuppression, or for promoting tissue repair and/or tissue regeneration, wherein the method comprises administering a cell, particularly a Treg cell, a cell population, or a pharmaceutical composition, particularly comprising a Treg, according to the invention.

In this respect, the method comprises the following steps:
(i) isolation or provision of a Treg-enriched cell sample from a subject;
(ii) introduction into the Treg cells of a nucleic acid molecule or vector of the invention; and
(iii) administering the Treg cells from (ii) to the subject.

The neurodegenerative disease may be amyotrophic lateral sclerosis (ALS), frontotemporal dementia (FTD), progressive supranuclear palsy (PSP), Parkinson's disease or Alzheimer's disease. Preferably, the disease may be ALS.

The invention also provides use of a cell, cell population or pharmaceutical composition according to the invention in the manufacture of a medicament for treating and/or preventing a neurological disease, disorder or injury, such as a neurodegenerative disease, or autoimmune or inflammatory disease, or for inducing immunosuppression, or for promoting tissue repair and/or tissue regeneration in a subject, particularly wherein the cell is a Treg cell.

In another aspect, the invention provides a method of making a cell according to the invention, which comprises the step of introducing into the cell (e.g., transducing or transfecting a cell with) the nucleic acid molecule or vector according to the invention. The cell may be a Treg cell, and the method may comprise isolating or providing a cell-containing sample comprising Tregs, and/or enriching Tregs or generating Tregs from the cell-containing sample prior to or after the step of introducing the nucleic acid molecule or vector into the cell. The invention also provides a cell obtainable by this method, which forms a further aspect of the invention.

In a further aspect, the invention provides the use of a CAR-Treg for inducing an anti-inflammatory microglial phenotype. Alternatively viewed, the invention provides the use of a CAR-Treg for inducing an M2 microglial phenotype. The CAR may be the CAR of present invention, i.e., it may comprise an antigen recognition domain that specifically binds to TREM2 (e.g., to human TREM2), and it may have any of the features of the CAR as disclosed herein.

In another aspect, the invention provides the use of a CAR-Treg for increasing the number of microglial cells expressing the anti-inflammatory marker arginase-1 (ARG1). The CAR may be the CAR of present invention, i.e., it may comprise an antigen recognition domain that specifically binds to TREM2 (e.g., to human TREM2), and it may have any of the features of the CAR as disclosed herein.

### FIGURES

FIG. 1 shows expression of constructs 3, 7, 8 and 10 in Jurkat NFAT cells pre-sort (FIG. 1a) and post-sort (FIG. 1b). Constructs 3, 7, 8 and 10 are all constructs of the present invention, which comprise a CAR comprising a TREM2 scFv. Non-transduced cells (mock) and transduced cells were collected and stained for flow cytometry using Biotinylated Human TREM2 Protein (TR2-H82E7 ACRObiosystems) and Streptavidin APC as a secondary antibody. Cells were sorted using Streptavidin magnetic beads, columns (miltenyi) and Biotinylated Human TREM2 Protein (TR2-H82E7 ACRObiosystems). The Y axis shows expression of GFP and the X axis shows expression of TREM2.

FIG. 2 shows the results of an activation assay, in which the Jurkat NFAT cells expressing constructs 3, 7, 8 and 10 were either non-stimulated, activated with OKT3 as a positive control (anti CD3 antibody for activation through endogenous TCR expressed by Jurkat cells) or activated with various TREM2 proteins.

FIG. 3 shows target cell lines that were engineered to over-express TREM2, namely, SUPT1 cells, HEK293t cells and THP1 cells. The Y axis shows expression of GFP and the X axis shows expression of TREM2. These cell lines were used in the experiments shown in FIG. 4.

FIG. 4 shows the results of an activation assay, in which the Jurkat NFAT cells expressing constructs 3, 7, 8 and 10 were either non-stimulated, activated with OKT3 as a positive control, activated with the cell lines over-expressing TREM2 (i.e., with the SUPT1 cells, HEK293t cells and THP1 cells shown in FIG. 3) or contacted with mock cell lines (i.e., with non-transduced SUPT1 cells, HEK293t cells or THP1 cells).

FIG. 5 shows similar results to those shown in FIGS. 2 and 4 but presented in a different way. It shows the results of an activation assay in which Jurkat NFAT cells expressing constructs 3, 7, 8 and 10 were either non-stimulated, activated with OKT3 as a positive control, activated with a biotinylated human TREM2 peptide with a streptavidin tag, or a mouse TREM2 peptide with a His-tag, or activated with a HEK293T cell line over-expressing human or murine TREM2, or contacted with a non-transduced HEK293T cell line as a negative control.

FIG. 6 shows expression of TREM2 in cell lines that were engineered to over-express TREM2 (namely HEK293 transduced to express human or mouse TREM2) and in THP-1 cells, which naturally express TREM2. Non-transduced cells (HEK293) or FMO controls (THP-1) were used as negative controls. These cell lines were used in the experiments shown in FIG. 8.

FIG. 7 shows cumulative data and representative data of the expression of constructs 3, 7, 8 and 10 in murine Treg cells. “Mock” is non-transduced cells. The Y axis shows expression of GFP and the X axis shows expression of TREM2.

FIG. 8 shows the results of an activation assay in which murine Treg cells expressing constructs 3, 7, 8 and 10 were activated with human or murine TREM2 peptides, HEK293 cell lines over-expressing human or murine TREM2, THP-1 cells or anti-CD3/anti-CD28 beads (as a positive control). Non-transduced HEK293 cells (“HEK293 WT”) and media alone (“unstimulated”) were used as negative controls. After 24 h, murine Tregs were analysed for the expression of activation markers: CD137, CD69 and CD44.

FIG. 9 shows the results of an in vivo trafficking/activation assay, where accumulation and activation of non-transduced CD45.1 murine T effector cells (“mock”), transduced CD45.1 murine T effector cells (“TREM2”, i.e. an anti-TREM2 CAR construct in accordance with the present invention which expresses GFP, namely construct 8) and a GFP control (“GFP mock”) in the central nervous system (CNS) of SOD1 mice and wild-type (WT) mice was assessed at day 6 after intrathecal delivery. The CD45.1+/GFP+ cells are the injected cells that have been successfully transduced with the anti-TREM2 CAR construct and are therefore specific for the TREM2 target.

FIG. 10 shows the results of an in vivo trafficking assay, where accumulation of transduced CD45.1 murine T effector cells (“TREM2 CAR T cells”, i.e. an anti-TREM2 CAR construct in accordance with the present invention which expresses GFP, namely construct 8) or a GFP control (“GFP+ T-cells”) in the spinal cord of EAE mice (“immunized”) and wild-type mice (“non-immunized”) was assessed at day 16 after intravenous delivery. The graph on the left shows the percentage of CD45.1+ cells in the spinal cord, whereas the graph on the right shows the percentage of those CD45.1+ cells that are also GFP+ (i.e. that have been successfully transduced with the anti-TREM2 CAR construct and are therefore specific for the TREM2 target).

FIG. 11 shows the progression of experimental autoimmune encephalomyelitis (EAE) disease in EAE mice as compared to wild-type mice, wherein the mice have been injected with murine T effector cells that have been transduced with a TREM2 CAR (i.e., an anti-TREM2 CAR construct in accordance with the present invention which expresses GFP (construct 8)) or with a GFP control. EAE is the most used experimental model for multiple sclerosis, i.e., a neuro-inflammatory disease. Disease progression is measured by clinical score, where 0 is no disease and 5 is peak disease/death (see Table 2 in the Examples).

FIG. 12 is a diagram showing the set-up of a contact-free co-culture experiment using mouse Treg cells and mouse BV2 microglial cell lines.

FIG. 13 shows results from the co-culture experiment depicted in FIG. 12. Mouse Tregs promote the induction of an anti-inflammatory profile in the BV2 murine microglial cells after 24 h of co-culture. The anti-inflammatory phenotype is identified by the increase in the frequency (%) (FIG. 13a) and number (#) (FIG. 13b) of ARG1+ microglial cells following co-culture with Tregs.

### DETAILED DESCRIPTION

The present invention provides CAR-Tregs specific for TREM2 that are activated in the presence of TREM2 antigen which is expressed in the CNS. Thus, these CAR-Tregs have therapeutic potential in treating neurological diseases, disorders or injury, such as neurodegenerative diseases, or autoimmune and inflammatory diseases. In particular, these engineered Tregs have therapeutic potential in ALS.

Accordingly, the present invention provides a chimeric antigen receptor (CAR) comprising an antigen recognition domain that binds to TREM2, a cell or cell population expressing the CAR (e.g., a Treg) and use of the cell or cell population in treating certain diseases, e.g., neurodegenerative diseases such as ALS.

A “Chimeric antigen receptor”, “CAR” or “CAR construct” refers to engineered receptors which can confer an antigen specificity onto cells (e.g., immune cells, such as Tregs). In particular, a CAR enables a cell to bind specifically to a particular antigen, e.g., a target molecule such as a target protein, whereupon a signal is generated by the endodomain (comprising an intracellular signalling domain) of the CAR, e.g., a signal resulting in activation of the cell. CARs are also known as artificial T-cell receptors, chimeric T-cell receptors or chimeric immunoreceptors.

The structure of CARs is well-known in the art and several generations of CARs have been produced. For instance, as a minimum a CAR may contain an extracellular antigen-specific targeting region, antigen binding domain, target binding domain or ligand binding domain, which is or forms part of the exodomain (also known as the extracellular domain or ectodomain) of the CAR, a transmembrane domain, and an intracellular signalling domain (which is, or is comprised within, an endodomain). However, the CAR may contain further domains to improve its functionality, e.g., one or more co-stimulatory domains to improve T cell proliferation, cytokine secretion, resistance to apoptosis, and in vivo persistence.

Thus, a chimeric receptor or CAR construct generally comprises a binding domain (which may be viewed as an antigen (i.e., target) or ligand binding domain and the terms binding domain, antigen binding domain and ligand binding domain are used interchangeably herein), optionally a hinge domain, which functions as a spacer to extend the binding domain away from the plasma membrane of the cell (e.g., immune cell) on which it is expressed, a transmembrane domain, an intracellular signalling domain (e.g., the signalling domain from the zeta chain of the CD3 molecule (CD3ζ) of the TcR complex, or an equivalent) and optionally one or more co-stimulatory domains, which may assist in signalling or functionality of the cell expressing the CAR. A CAR may also comprise a signal or leader sequence or domain which functions to target the protein to the membrane and may form part of the exodomain of the CAR. The different domains may be linked directly or by linkers, and/or may occur within different polypeptides, e.g., within two polypeptides which associate with one another.

When the CAR binds its target antigen (i.e., TREM2), this results in the transmission of an activating signal to the cell in which it is expressed. Thus, the CAR directs the specificity of the engineered cells towards TREM2, particularly towards cells expressing TREM2.

The term “directed towards” or “directed against” is synonymous with “specific for” or “anti”. Put another way, the CAR recognises the TREM2 target molecule. Accordingly, it is meant that the CAR is capable of binding specifically to TREM2. In particular, the antigen-binding domain of the CAR is capable of binding specifically to TREM2 (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing TREM2, particularly a target cell expressing TREM2 on its cell surface.

The human isoform 1 of TREM2 (SEQ ID NO: 68) comprises an exodomain (amino acids 1-174 of SEQ ID NO: 68), a transmembrane domain (amino acids 175-195 of SEQ ID NO: 68) and an endodomain (amino acids 196-230 of SEQ ID NO: 68). The exodomain of TREM2 (SEQ ID NO: 69) comprises at least three domains: a signal or leader sequence (amino acids 1-18 of SEQ ID NO: 69); a ligand binding domain (amino acids 19-130 of SEQ ID NO: 69); and a stalk region (amino acids 131-174 of SEQ ID NO: 69). The ligand binding domain and the stalk region have a sequence as set out in SEQ ID NO. 70 (amino acids 19-174 of SEQ ID NO. 69). The ligand binding domain of the exodomain comprises three complementary determining regions (CDR1: amino acids 38-47 of SEQ ID NO: 68; CDR2: amino acids 65-75 of SEQ ID NO: 68; and CDR3: amino acids 88-91 of SEQ ID NO: 68).

The exodomain of TREM2 also comprises a dipeptide sheddase cleavage site (amino acids 157-158 of SEQ ID NO: 68), which is cleaved by a Disintegrin and metalloproteinase domain-containing protein (ADAM) 10 and, to a lesser extent, ADAM17. The exodomain of TREM2 may further comprise a meprin beta cleavage site between amino acids 136-137 of SEQ ID NO. 68.

When cells expressing the CARs of the present invention bind to TREM2, they may have the ability to stabilise the TREM2 receptor and prevent cleavage of the ligand binding extracellular domain.

TREM2 has a variety of ligands that bind its ligand binding domain (e.g., amino acid residues 19-130 of SEQ ID NO: 69). The terms “TREM2 ligand” or “TREM2-L” refer to any ligand which binds specifically to TREM2 via its extracellular domain, particularly via its ligand binding domain. TREM2 has been reported to bind to phosphatidylserine (PS), phosphatidylethanolamine (PE), phosphatidylcholine (PC), sulfoglycolipid, apolipoproteins (e.g., ApoE), low-density lipoprotein, high-density lipoprotein, heat shock protein 60, DNA, E. coli, apoptotic cells, and Amyloid p peptide. It will be understood by a skilled person that a CAR expressing any TREM2 ligand, or a part thereof, could be used to target TREM2.

TREM2 is expressed on cells associated with autoimmune diseases and inflammatory diseases (e.g., on microglial cells). It will be understood by a skilled person that where the cell engineered to express the CAR of the present invention is a Treg cell, or a precursor thereof, due to the bystander effect of Treg cells, the antigen may be simply present and/or expressed at the site of inflammation or disease.

The antigen-binding domain of a CAR may be derived or obtained from any protein or polypeptide which binds (i.e., has affinity for) TREM2. This may be for example, a ligand of TREM2 as mentioned above, or a physiological binding protein for TREM2, or a part thereof, or a synthetic or derivative protein. The target molecule (i.e., TREM2) may commonly be expressed on the surface of a cell, for example a target cell (e.g., a microglial cell), or a cell in the vicinity of a target cell (for a bystander effect), but need not be. Depending on the nature and specificity of the antigen binding domain, the CAR may recognise a soluble molecule, i.e., the cleaved extracellular domain of TREM2.

The antigen-binding domain is most commonly derived from antibody variable chains (for example it commonly takes the form of a scFv), but may also be generated from other molecules, such as ligands or other binding molecules.

The CAR is typically expressed as a polypeptide also comprising a signal sequence (also known as a leader sequence), and in particular a signal sequence which targets the CAR to the plasma membrane of the cell. This will generally be positioned next to or close to the antigen-binding domain, generally upstream of the antigen-binding domain. The extracellular domain, or ectodomain, of the CAR may thus comprise a signal sequence and an antigen-binding domain.

As noted above, the antigen-binding domain may be any protein or peptide that possesses the ability to specifically recognize and bind to TREM2. The antigen-binding domain includes any naturally occurring, synthetic, semi-synthetic, or recombinantly produced binding partner for TREM2. Illustrative antigen-specific targeting domains include antibodies or antibody fragments or derivatives, or ligands for soluble or membrane bound TREM2.

In an embodiment, the antigen binding domain is, or is derived from, an antibody. An antibody-derived binding domain can be a fragment of an antibody or a genetically engineered product of one or more fragments of the antibody, which fragment is involved in binding with the antigen. Examples include a variable region (Fv), a complementarity determining region (CDR), Fab or F(ab′)₂, or the light and heavy chain variable regions can be joined together in a single chain (e.g., as a scFv) and in either orientation (e.g. V_{L}-V_{H} or V_{H}-V_{L}). The V_{L} and/or V_{H} sequences may be modified. In particular the framework regions may be modified (e.g., substituted, for example to humanise the antigen-binding domain). Other examples include a heavy chain variable region (VH), a light chain variable region (VL) a camelid antibody (VHH) and a single domain antibody (sAb).

In a preferred embodiment, the binding domain is a single chain antibody (scFv). The scFv may be murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another, in either orientation, directly or via a peptide linker sequence.

Antibodies that specifically bind a predetermined antigen, i.e., TREM2, can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule, i.e., to TREM2. Phage display libraries of human antibodies are also available. Once identified, the amino acid sequence or polynucleotide sequence coding for the antibody can be isolated and/or determined.

The antigen recognition domain may bind, suitably specifically bind, one or more regions or epitopes within TREM2. An epitope, also known as antigenic determinant, is the part of an antigen that is recognised by an antigen recognition domain (e.g., an antibody). In other words, the epitope is the specific piece of the antigen to which an antibody binds. Suitably, the antigen recognition domain binds, suitably specifically binds, to one region or epitope within TREM2.

The antigen recognition domain may comprise at least one CDR (e.g. CDR3), which can be predicted from an antibody which binds to an antigen, i.e., TREM2 (or a variant of such a predicted CDR (e.g. a variant with one, two or three amino acid substitutions)). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13: 1882-91).

In this respect, the antigen binding domain may comprise one or more variable heavy chain CDRs, e.g., one, two or three variable heavy chain CDRs. Alternatively, or additionally, the antigen binding domain may comprise one or more variable light chain CDRs, e.g. one, two or three variable light chain CDRs. The antigen binding domain may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an antibody which binds to TREM2.

The antigen binding domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the antigen binding domain comprises three variable heavy chain CDRs (CDR1, CDR2 and CDR3) and/or three variable light chain CDRs (CDR1, CDR2 and CDR3).

The one or more CDRs present within the antigen binding domain may not all be from the same antibody, as long as the domain has the desired binding activity. Thus, one CDR may be predicted from the heavy or light chains of an antibody which binds to TREM2 whilst another CDR present may be predicted from a different antibody which binds to TREM2. A combination of CDRs may be used from different antibodies, particularly from antibodies that bind to the same desired region or epitope.

In a particularly preferred embodiment, the antigen binding domain comprises three CDRs predicted from the variable heavy chain sequence of an antibody which binds to TREM2 and/or three CDRs predicted from the variable light chain sequence of an antibody which binds to TREM2 (preferably the same antibody).

In an embodiment, the antigen-binding domain comprises VH CDR1, 2 and 3 sequences as set forth in SEQ ID NOs. 1, 2 and 3 respectively and VL CDR1, 2 and 3 sequences as set forth in SEQ ID NOs. 4, 5 and 6 respectively, or the CDRs may contain 1 to 3, or more particularly 1 or 2 amino acid sequence modifications to the CDR sequences set out in any aforementioned sequence.

More particularly, in such an embodiment the antigen binding domain of the CAR comprises a VH domain comprising the sequence as set forth in SEQ ID NO. 25, or a sequence having at least 70% sequence identity thereto, and a VL domain comprising the sequence as set forth in SEQ ID NO. 26, or a sequence having at least 70% sequence identity thereto.

In another embodiment, the antigen-binding domain comprises VH CDR1, 2 and 3 sequences as set forth in SEQ ID NOs. 7, 8 and 9 respectively and VL CDR1, 2 and 3 sequences as set forth in SEQ ID NOs. 10, 11, and 12 respectively, or the CDRs may contain 1 to 3, or more particularly 1 or 2 amino acid sequence modifications to the CDR sequences set out in any aforementioned sequence.

More particularly, in such an embodiment the antigen binding domain of the CAR comprises a VH domain comprising the sequence as set forth in SEQ ID NO. 27, or a sequence having at least 70% sequence identity thereto, and a VL domain comprising the sequence as set forth in SEQ ID NO. 28, or a sequence having at least 70% sequence identity thereto.

In another embodiment, the antigen-binding domain comprises VH CDR1, 2 and 3 sequences as set forth in SEQ ID NOs. 13, 14 and 15 respectively and VL CDR1, 2 and 3 sequences as set forth in SEQ ID NOs. 16, 17, and 18 respectively, or the CDRs may contain 1 to 3, or more particularly 1 or 2 amino acid sequence modifications to the CDR sequences set out in any aforementioned sequence.

More particularly, in such an embodiment the antigen binding domain of the CAR comprises a VH domain comprising the sequence as set forth in SEQ ID NO. 29, or a sequence having at least 70% sequence identity thereto, and a VL domain comprising the sequence as set forth in SEQ ID NO. 30, or a sequence having at least 70% sequence identity thereto.

In another embodiment, the antigen-binding domain comprises VH CDR1, 2 and 3 sequences as set forth in SEQ ID NOs. 19, 20 and 21 respectively and VL CDR1, 2 and 3 sequences as set forth in SEQ ID NOs. 22, 23, and 24 respectively, or the CDRs may contain 1 to 3, or more particularly 1 or 2 amino acid sequence modifications to the CDR sequences set out in any aforementioned sequence.

More particularly, in such an embodiment the antigen binding domain of the CAR comprises a VH domain comprising the sequence as set forth in SEQ ID NO. 31, or a sequence having at least 70% sequence identity thereto, and a VL domain comprising the sequence as set forth in SEQ ID NO. 32, or a sequence having at least 70% sequence identity thereto. Where a CDR does contain an amino acid sequence modification, this may be a deletion, addition, or substitution of an amino acid residue of the CDR sequence as set out in the above-mentioned SEQ ID NOs. More particularly, the modification may be an amino acid substitution, for example a conservative amino acid substitution, e.g., as set out above. A longer CDR may tolerate more amino acid residue modifications. In the case of CDRs which are 5 or 7 amino acid residues long, the modifications may be of 0, 1, 2 or 3 residues, e.g. 2 residues. In general, there may be 0, 1, 2, or 3 modifications to any particular CDR sequence. Further, in an embodiment, CDRs 1 and 2 may be modified, and CDR3 may be unmodified. In another embodiment all 3 CDRs may be modified. In another embodiment, the CDRs are not modified.

The antigen binding domain may be in the form of a scFV comprising the VH and VL domain sequences as set out above, in either order, for example VH-VL. The VH and VL sequences may be linked by a linker sequence.

In this respect, in one embodiment, the antigen binding domain of the CAR comprises the sequence as set forth in SEQ ID NO: 33 or a sequence having at least 80% identity thereto.

In another embodiment, the antigen binding domain of the CAR comprises the sequence as set forth in SEQ ID NO: 34 or a sequence having at least 80% identity thereto.

In a further embodiment, the antigen binding domain of the CAR comprises the sequence as set forth in SEQ ID NO: 35 or a sequence having at least 80% identity thereto.

In another embodiment, the antigen binding domain of the CAR comprises the sequence as set forth in SEQ ID NO: 36 or a sequence having at least 80% identity thereto.

Suitable linkers can be readily selected and can be of any of a suitable length, such as from 1 amino acid (e.g. Gly) to 30 amino acids, e.g. from any one of 2, 3, 4, 5, 6, 7, 8, 9, or 10 amino acids to any one of 12, 15, 18, 20, 21, 25, 30 amino acids, for example, 5-30, 5-25, 6-25, 10-15, 12-25, 15 to 25 etc.

The linker may for example be a linker as discussed below in relation to the safety switch polypeptide. Exemplary flexible linkers include glycine polymers (G), glycine-serine polymers, where n is an integer of at least one, glycine-alanine polymers, alanine-serine polymers, and other flexible linkers known in the art, as discussed above. The linker may comprise 1 or more “GS” domains as discussed above.

Accordingly, in one embodiment the antigen binding domain may comprise, or consist of, a VH sequence as set forth in SEQ ID NO. 25 linked via a linker of sequence (X)n, where X is any amino acid and n is an integer of between 15 and 25, to the VL sequence as set forth in SEQ ID NO. 26. The antigen binding domain may comprise or consist of a sequence which is a variant of SEQ ID NO. 33, which has at least 70% sequence identity thereto.

In another embodiment the antigen binding domain may comprise, or consist of, a VH sequence as set forth in SEQ ID NO. 27 linked via a linker of sequence (X)n, where X is any amino acid and n is an integer of between 15 and 25, to the VL sequence as set forth in SEQ ID NO. 28. The antigen binding domain may comprise or consist of a sequence which is a variant of SEQ ID NO. 34, which has at least 70% sequence identity thereto.

In a further embodiment the antigen binding domain may comprise, or consist of, a VH sequence as set forth in SEQ ID NO. 29 linked via a linker of sequence (X)n, where X is any amino acid and n is an integer of between 15 and 25, to the VL sequence as set forth in SEQ ID NO. 30. The antigen binding domain may comprise or consist of a sequence which is a variant of SEQ ID NO. 35, which has at least 70% sequence identity thereto.

In another embodiment the antigen binding domain may comprise, or consist of, a VH sequence as set forth in SEQ ID NO. 31 linked via a linker of sequence (X)n, where X is any amino acid and n is an integer of between 15 and 25, to the VL sequence as set forth in SEQ ID NO. 32. The antigen binding domain may comprise or consist of a sequence which is a variant of SEQ ID NO. 36, which has at least 70% sequence identity thereto.

The variant sequences disclosed and described herein, including the variant VH, VL and antigen binding domain sequences, may have at least 75, 80, 85, 90, 92, 95, 96, 97, 98, or 99% sequence identity to the specified SEQ ID NOs.

The CAR also preferably comprises a hinge domain to hold the extracellular domain, particularly the antigen binding domain, away from the cell surface, and comprises a transmembrane domain. The hinge and transmembrane domains may comprise the hinge and transmembrane sequences from any protein which has a hinge domain and/or a transmembrane domain, including any of the type 1, type II or type Ill transmembrane proteins. The hinge domain may be selected from the hinge regions of CD28, CD8alpha, CD4, CD7, CH2CH3, an immunoglobulin, or a part or variant thereof. Typically, the hinge may be derived from CD8, particularly, CD8alpha, or from CH2CH3. Preferably, the hinge may be derived from CD8alpha.

The transmembrane domain of the CAR may also comprise an artificial hydrophobic sequence. The transmembrane domains of the CAR may be selected so as not to dimerize.

Additional transmembrane domains will be apparent to those of skill in the art. Examples of transmembrane (TM) regions used in CAR constructs are: 1) The CD28 TM region (Pule et al, Mol Ther, 2005, November; 12(5):933-41; Brentjens et al, CCR, 2007, Sep. 15; 13(18 Pt 1):5426-35; Casucci et al, Blood, 2013, Nov. 14; 122(20):3461-72); 2) The OX40 TM region (Pule et al, Mol Ther, 2005, November; 12(5):933-41); 3) The 41BB TM region (Brentjens et al, CCR, 2007, Sep. 15; 13(18 Pt 1):5426-35); 4). The CD3 zeta TM region (Pule et al, Mol Ther, 2005, November; 12(5):933-41; Savoldo B, Blood, 2009, Jun. 18; 113(25):6392-402); 5) The CD8α TM region (Maher et al, Nat Biotechnol, 2002, January; 20(1):70-5; Imai C, Leukemia, 2004, April; 18(4):676-84; Brentjens et al, CCR, 2007, Sep. 15; 13(18 Pt 1):5426-35; Milone et al, Mol Ther, 2009, August; 17(8):1453-64). Other transmembrane domains which may be used include those from ICOS, CD4, CD45, CD9, CD16, CD22, CD33, CD64, CD80, CD86, CD154 or CH2CH3. Preferably, the transmembrane domain may be derived from CD8α or CH2CH3.

A hinge domain may conveniently be obtained from the same protein as the transmembrane domain. In one embodiment, where the transmembrane domain is derived from the CD8α transmembrane domain, the hinge domain is derived from the CD8α hinge domain. In an alternative embodiment, where the transmembrane domain is derived from the CH2CH3 transmembrane domain, the hinge domain is derived from the CH2CH3 hinge domain.

The CD8α hinge domain may be combined with a CD8α transmembrane domain. In an embodiment, the CAR comprises a CD8α hinge and transmembrane domain sequence shown as SEQ ID NO: 43, or a variant which is at least 80% identical to SEQ ID NO: 43. Suitably, the variant may be at least 85, 90, 95, 97, 98 or 99% identical to SEQ ID NO: 43.

Alternatively, the CAR may comprise a domain derived from the CH2CH3 transmembrane domain, which may be combined with a domain derived from a CH2CH3 hinge domain.

Alternatively, a CD28 hinge and transmembrane sequence may be used or a variant thereof.

By way of further example, the CAR may comprise a native or modified CD8α hinge domain and a CD28 transmembrane domain, or a CD28 hinge domain and CD8α transmembrane domain.

Other hinge domains which may be used include those from CD4, CD7, or an immunoglobulin, or a part or variant thereof.

The CAR may further comprise a signal (or alternatively termed, leader) sequence which targets it to the endoplasmic reticulum pathway for expression on the cell surface. An illustrative signal/leader sequence is MASPLTRFLSLNLLLLGESIILGSGEA as shown in SEQ ID NO. 42, or a variant sequence having at least 70% sequence identity thereto may be used.

The endodomain of a CAR as described herein comprises motifs necessary to transduce the effector function signal and direct a cell expressing the CAR to perform its specialized function upon antigen binding. Particularly, the endodomain may comprise one or more (e.g., two or three) Immunoreceptor tyrosine-based activation motifs (ITAMs), typically comprising the amino acid sequence of YXXL/1, where X can be any amino acid. Examples of intracellular signaling domains include, but are not limited to, ζ chain endodomain of the T-cell receptor or any of its homologs (e.g., η chain, FcεR1γ and β chains, MB1 (Igα) chain, B29 (Igβ) chain, etc.), CD3 polypeptide domains (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human or mouse CD3 zeta chain endodomain, FcγRIII, FcsRI, cytoplasmic tails of Fc receptors, immunoreceptor tyrosine-based activation motif (ITAM) bearing cytoplasmic receptors or combinations thereof.

Commonly, the intracellular signaling domain comprises the intracellular signaling domain of a CD3 zeta chain. The sequence of the intracellular signaling domain of murine CD3 zeta chain is set out in SEQ ID NO. 45. The CAR may comprise a CD3ζ signalling domain comprising or consisting of a sequence as set out in SEQ ID NO. 45 or a sequence having at least 80, 85, 90, 95, 97, 98 or 99% identity to SEQ ID NO: 45. In an embodiment the signaling domain comprises or consists of SEQ ID NO. 45.

Other signaling domains which may be used include the signaling domains of CD28 or CD27 or variants thereof. Additional intracellular signaling domains will be apparent to those of skill in the art and may be used in connection with alternate embodiments of the invention. In one embodiment, the present CAR may not comprise a costimulatory domain derived from 41 BB within the endodomain.

The present CAR may comprise a compound endodomain comprising a fusion of the intracellular part of a T-cell co-stimulatory molecule to that of e.g. CD3. Such a compound endodomain may be referred to as a second-generation CAR which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. The CAR endodomain may also comprise one or more TNF receptor family signalling domain, such as the signalling domain of ICOS, (CD134) OX40, 4-1BB, CD27 or TNFRSF25, or a part or variant thereof, although preferably the CAR may not comprise an endodomain comprising the signalling domains of both CD28 and 41BB.

The intracellular signaling domain of CD28 which may be used as a co-stimulatory domain is shown in SEQ ID NO. 44. Illustrative sequences for OX40, 4-1 BB, ICOS, and TNFRSF25 signalling domains which are shown in SEQ ID NO: 50 to 53 respectively. The CAR may comprise one or more co-stimulatory domains comprising or consisting of the sequence of any one of SEQ ID NO: 44, 50, 51, 52 or 53, or a variant thereof having at least 80, 85, 90, 95, 97, 98 or 99% sequence identity thereto.

In an embodiment the CAR comprises a co-stimulatory domain comprising or consisting of SEQ ID NO. 44.

In one embodiment the CAR comprises a human CD8 hinge domain or a variant thereof and a human CD8 transmembrane domain. Alternatively, or additionally, the CAR comprises an endodomain comprising a human CD28 co-stimulatory domain and a human CD3ζ signalling domain.

In one preferred embodiment the CAR comprises a hinge, transmembrane, and intracellular (or endo) domains as follows:
(i) a CD8α hinge and transmembrane domain sequence comprising or consisting of the sequence as set forth in SEQ ID NO. 43, or a sequence having at least 80% sequence identity thereto;
(ii) a CD28 co-stimulatory domain comprising or consisting of the sequence as set forth in SEQ ID NO. 44, or a sequence having at least 80% sequence identity thereto;
(iii) a CD3ζ signalling domain comprising or consisting of the sequence as set forth in SEQ ID NO. 45, or a sequence having at least 80% sequence identity thereto.

The CAR, as encoded and expressed, may further comprise a leader sequence comprising or consisting of a sequence as set out in SEQ ID NO. 42, or a sequence having at least 80% sequence identity thereto.

The antigen binding domain of the CAR may comprise or consist of a sequence as set out in SEQ ID NO. 33, 34, 35 or 36, or a sequence having at least 80% sequence identity thereto which is capable of binding to TREM2.

Thus, in its entirety one preferred representative CAR may comprise:
(i) a leader sequence comprising or consisting of a sequence as set out in SEQ ID NO. 42, or a sequence having at least 80% sequence identity thereto;
(ii) an antigen binding domain comprising or consisting of a sequence as set out in SEQ ID NO. 33, 34, 35 or 36, or a sequence having at least 80% sequence identity thereto;
(iv) a CD8α hinge and transmembrane domain sequence comprising or consisting of the sequence as set forth in SEQ ID NO. 43, or a sequence having at least 80% sequence identity thereto;
(v) a CD28 co-stimulatory domain comprising or consisting of the sequence as set forth in SEQ ID NO. 44, or a sequence having at least 80% sequence identity thereto;
(vi) a CD3ζ signalling domain comprising or consisting of the sequence as set forth in SEQ ID NO. 45, or a sequence having at least 80% sequence identity thereto.

The CAR is capable of binding to TREM2 and of transducing a signal into a cell in which it is expressed. The cell may then be activated and may exert a suppressive effect within the local environment. Activation of a cell expressing a CAR after antigen binding can be determined by an increased level of CD69 as compared to the same cells expressing a CAR in the absence of antigen. For example, an increase of at least 10, 20, 30, 40, 50, 60, 70, 80 or 90% in CD69. Expression levels of CD69 can be determined using standard techniques, for example FACS, using commercially available antibodies (e.g. FITC anti-human CD69 antibody, Biolegend). Thus, CAR function within a cell can be determined by activation status of the cell in which the CAR is expressed, e.g., by determining CD69 expression.

The endodomain of a CAR herein may contain further domains. For example, it may comprise a domain comprising a STAT5 association motif, a JAK1 and/or JAK 2 binding motif and optionally a JAK 3 binding motif. In such an embodiment the endodomain may comprise one or more sequences from an endodomain of a cytokine receptor, for example an interleukin receptor (IL) receptor. Such CARs are described in WO2020/044055 (also incorporated herein by reference). The inclusion of such domains confers the ability on the CAR to provide a productive IL signal to the cell in which it is expressed in an antigen-specific manner, without requiring exogenous IL to be administered. For example, IL-2 is important for the survival, proliferation and persistence of Treg cells, but IL-2 levels may frequently be low or impaired in patients needing treatment. A CAR may thus comprise a sequence corresponding to all or part of a p chain endodomain of an IL receptor or a variant thereof, such as the IL2 receptor, optionally in combination with the γ chain endodomain of an IL receptor or a variant thereof, such as the IL2 receptor.

By way of example, the CAR endodomain may comprise a domain comprising a sequence from the human IL-2 receptor B chain, or a variant thereof, as follows:
a sequence as set forth in, SEQ ID NO. 54, which represents amino acid numbers 266 to 551 of human IL-2 receptor p chain (NCBI REFSEQ: NP_000869.1), or a sequence with at least 80% sequence identity to SEQ ID NO. 54; or
a sequence as set forth in SEQ ID NO.55, which represents a truncated and sequence modified variant of SEQ ID NO. 54 (substitution Y510), or a sequence with at least 80% sequence identity to SEQ ID NO. 55; or
a sequence as set forth in SEQ ID NO. 56, which represents a truncated and sequence modified variant of SEQ ID NO. 54 (substitutions Y510 and Y392), or a sequence with at least 80% sequence identity to SEQ ID NO. 56.

As mentioned above, the cell or cell population of the invention may further comprise additional polypeptides, particularly exogenous polypeptides, such as a FOXP3 and/or safety switch polypeptide. The polypeptides of the present invention, e.g., the CAR, FOXP3 and safety switch, may be encoded by a single nucleic acid molecule. The nucleic acid molecule may comprise nucleotide sequences encoding self-cleavage sequences in between the encoded polypeptides, allowing the polypeptides to be expressed and/or produced as separate, or discrete components. By this it is meant that, although the polypeptides are encoded by a single nucleic acid molecule, through “cleavage” during or after translation at the encoded cleavage sites, they may be expressed or produced as separate polypeptides, and thus at the end of the protein production process in the cell, they may be present in the cell as separate entities, or separate polypeptide chains. Alternatively, additional exogenous polypeptides may be encoded by separate nucleic acid molecules or vectors.

By “discrete” or “separate” polypeptides it is meant that the polypeptides are not linked to one another and are physically distinct. Indeed, following expression, they are located in different, or separate cellular locations. The CAR, FOXP3 and safety switch polypeptide are thus ultimately expressed as single and separate components. The CAR is expressed as a cell surface molecule. The safety switch polypeptide may be expressed inside a cell, or on the cell surface. In a particular embodiment, the safety switch polypeptide and the CAR are expressed on the surface of a cell which is intended for ACT. The FOXP3 is expressed inside the cell, where it can exert its effect as a transcription factor to regulate cell development and/or activity, as described further below.

The safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, in this case the CAR, which when expressed allows the cell to be deleted to turn off expression of the transgene (CAR). A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9. However, it is preferred for the suicide moiety to be, or to comprise, an epitope which is recognised by a cell-deleting antibody or other binding molecule capable of eliciting deletion of the cell. In such an embodiment, the safety switch polypeptide is expressed on the surface of a cell.

The term “delete” as used herein in the context of cell deletion is synonymous with “remove” or “ablate” or “eliminate” The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. Biosimilars for Rituximab are available and may be used. A person of skill in the art is readily able to use routine methods to prepare an antibody having the binding specificity of Rituximab using the available amino acid sequences therefor.

CAR-cells specific for TREM2, which also express a safety switch polypeptide comprising this sequence can be selectively killed using the antibody Rituximab, or an antibody having the binding specificity of Rituximab. The safety switch polypeptide is expressed on the cell surface and when the expressed polypeptide is exposed to or contacted with Rituximab, or an antibody with the same binding specificity, death of the cell ensues.

Thus, Rituximab, or an antibody having the binding specificity thereof, may be provided for use in ACT in combination with a cell of the invention. The cell or nucleic acid or vector or construct for production of the cell and the Rituximab or equivalent antibody may be provided in a kit, or as a combination product.

For example, the suicide constructs of WO2013/153391 or WO2021239812 (both incorporated herein by reference) may be used in a cell or cell population (e.g., Treg or Treg population) as described herein.

The nucleic acid molecule of the present invention may be designed to increase FOXP3 expression in cells (e.g., Tregs) by introducing into the cells a nucleotide sequence encoding FOXP3, which term is synonymous with the term “a FOXP3 polypeptide”. The nucleic acid molecule, and constructs and vectors containing it, thus provide a means for increasing FOXP3 in a cell, e.g., in a Treg or a CD4+ cell.

“FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

“Increasing FOXP3 expression” means to increase the levels of FOXP3 mRNA and/or protein in a cell (or population of cells) in comparison to a corresponding cell which has not been modified (or population of cells) by introduction of the nucleic acid molecule, construct or vector. For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Preferably the cell is a Treg or the population of cells is a population of Tregs.

Suitably, the level of FOXP3 mRNA and/or protein in a modified cell (or a population of such cells) may be increased to at least 1.5-fold greater, 2-fold greater, or 5-fold greater than the level in a corresponding cell which has not been so modified (or population of such cells). Preferably the cell is a Treg or the population of cells is a population of Tregs.

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e., a polypeptide able to bind FOXP3 target DNA and function as a transcription factor regulating development and function of Tregs. Particularly, a FOXP3 polypeptide may have the same or similar activity to wildtype FOXP3 (SEQ ID NO. 57), e.g., may have at least 40, 50, 60, 70, 80, 90, 95, 100, 110, 120, 130, 140 or 150% of the activity of the wildtype FOXP3 polypeptide. Thus, a FOXP3 polypeptide encoded by the nucleotide sequence in the nucleic acid, construct or vector described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference). As discussed in detail below, FOXP3 or a FOXP3 polypeptide includes functional fragments, variants, and isoforms thereof, e.g., of SEQ ID NO. 57.

A “functional fragment of FOXP3” may refer to a portion or region of a FOXP3 polypeptide or a polynucleotide (i.e., nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp. 665-675; Lopes, J. E., et al., 2006. The Journal of Immunology, 177(5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO. 61 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide, e.g., to SEQ ID NO. 57. FOXP3 variants may have the same or similar activity to a wildtype FOXP3 polypeptide or polynucleotide, e.g., may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g., at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO. 57, for example S418E and/or S422A, as described in WO2019/241549 (incorporated herein by reference) and are set out in SEQ ID NO.s 58 to 60, which represent the aa418, aa422 and aa418 and aa422 mutants respectively.

Suitably, the FOXP3 polypeptide encoded by a nucleic acid molecule, construct or vector as described herein may comprise or consist of the polypeptide sequence of a human FOXP3, such as UniProtKB accession Q9BZS1 (SEQ ID NO: 57), or a functional fragment or variant thereof.

In some embodiments of the invention, the FOXP3 polypeptide comprises or consists of an amino acid sequence which is at least 70% identical to SEQ ID NO: 57 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO: 57 or a functional fragment thereof. In some embodiments, the FOXP3 polypeptide comprises or consists of SEQ ID NO: 57 or a functional fragment thereof.

In some embodiments, as discussed above, the FOXP3 polypeptide may comprise mutations at residues 418 and/or 422 of SEQ ID NO. 57, as set out in SEQ ID NO. 58, SEQ ID NO. 59, or SEQ ID NO. 60.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of SEQ ID NO. 61 or a functional variant thereof having at least 80, 85, 90, 95 or 99% identity thereto.

Suitably, the FOXP3 polypeptide may be a variant of SEQ ID NO: 57, for example a natural variant. Suitably, the FOXP3 polypeptide is an isoform of SEQ ID NO: 57. For example, the FOXP3 polypeptide may comprise a deletion of amino acid positions 72-106 relative to SEQ ID NO: 57. Alternatively, the FOXP3 polypeptide may comprise a deletion of amino acid positions 246-272 relative to SEQ ID NO: 57.

Suitably, the FOXP3 polypeptide comprises SEQ ID NO: 62 or a functional fragment thereof. SEQ ID NO: 62 represents an Illustrative FOXP3 polypeptide.

Suitably the FOXP3 polypeptide comprises or consists of an amino acid sequence which is at least 70% identical to SEQ ID NO: 62 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 62 or a functional fragment thereof. In some embodiments, the FOXP3 polypeptide comprises or consists of SEQ ID NO: 62 or a functional fragment thereof.

Suitably, the FOXP3 polypeptide may be a variant of SEQ ID NO: 62, for example a natural variant. Suitably, the FOXP3 polypeptide is an isoform of SEQ ID NO: 62 or a functional fragment thereof. For example, the FOXP3 polypeptide may comprise a deletion of amino acid positions 72-106 relative to SEQ ID NO: 62. Alternatively, the FOXP3 polypeptide may comprise a deletion of amino acid positions 246-272 relative to SEQ ID NO: 62.

Suitably, the polynucleotide encoding a FOXP3 polypeptide comprises or consists of a nucleotide sequence set forth in SEQ ID NO: 63, which represents an illustrative FOXP3 nucleotide sequence.

In some embodiments of the invention, the polynucleotide encoding the FOXP3 polypeptide or variant comprises nucleotide sequence which is at least 70% identical to SEQ ID NO: 63 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 63 or a fragment thereof which encodes a functional FOXP3 polypeptide. In some embodiments of the invention, the polynucleotide encoding the FOXP3 polypeptide or variant comprises or consists of SEQ ID NO: 63 or a fragment thereof which encodes a functional FOXP3 polypeptide.

Suitably, the polynucleotide encoding a FOXP3 polypeptide comprises or consists of a polynucleotide sequence set forth in SEQ ID NO: 64, which represents another illustrative FOXP3 nucleotide.

In some embodiments of the invention, the polynucleotide encoding the FOXP3 polypeptide or variant comprises a nucleotide sequence which is at least 70% identical to SEQ ID NO: 64 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 64 or a fragment thereof which encodes a functional FOXP3 polypeptide. In some embodiments of the invention, the polynucleotide encoding the FOXP3 polypeptide or variant comprises or consists of SEQ ID NO: 64 or a fragment thereof which encodes a functional FOXP3 polypeptide.

A skilled person will appreciate that FOXP3 expression within a Treg may be increased indirectly by introducing a polynucleotide into the cell which encodes a protein which increases transcription and/or translation of FOXP3 or which increases the half-life (e.g., by at least 10, 20, 30, 40, 50, 60, 70, 80 or 90%) or function of FOXP3 (e.g. determined by suppressive ability of a transduced Treg, measured as previously discussed). For example, it may be possible to introduce a polynucleotide into a Treg which increases transcription of endogenous FOXP3 by interacting with the endogenous FOXP3 promoter or non-coding sequences (CNS, e.g., CNS1, 2 or 3) which are found upstream of the coding region.

Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

As mentioned above, the nucleic acid molecule may comprise nucleotide sequences encoding self-cleavage sequences. Particularly, the self-cleaving sequences are self-cleaving peptides. Such sequences auto-cleave during protein production. Self-cleaving peptides which may be used are 2A peptides or 2A-like peptides which are known and described in the art, for example in Donnelly et al., Journal of General Virology, 2001, 82, 1027-1041, herein incorporated by reference. 2A and 2A-like peptides are believed to cause ribosome skipping and result in a form of cleavage in which a ribosome skips the formation of peptide bond between the end of a 2A peptide and the downstream amino acid sequence.

The “cleavage” occurs between the Glycine and Proline residues at the C-terminus of the 2A peptide meaning the upstream cistron will have a few additional residues added to the end, while the downstream cistron will start with the Proline. The term “cleavage” as used herein thus includes the skipping of peptide bond formation.

Suitable self-cleaving domains include P2A, T2A, E2A, and F2A sequences as shown in SEQ ID NO: 46-49 respectively. The sequences may be modified to include the amino acids GSG at the N-terminus of the 2A peptides. Thus, also included as possible options are sequences corresponding to SEQ ID NOs. 46-49, but with GSG at the N termini thereof. Such modified alternative 2A sequences are known and reported in the art. Alternative 2A-like sequences which may be used are shown in Donnelly et al (supra), for example a TaV sequence.

The self-cleaving sequences included in the nucleic acid molecule may be the same or different. In an embodiment they are both 2A sequences, in particular P2A and/or T2A sequences.

The self-cleaving sequence may include an additional cleavage site, which may be cleaved by common enzymes present in the cell. This may assist in achieving complete removal of the 2A sequences after translation. Such an additional cleavage site may for example comprise a Furin cleavage site RXXR (SEQ ID NO: 65), for example RRKR (SEQ ID NO: 66).

In a representative embodiment, the nucleic acid molecule may comprise a nucleotide sequence encoding a CAR directed against TREM2 having the sequence of SEQ ID NO. 38, 39, 40 or 41, a nucleotide sequence encoding a safety switch and a nucleotide sequence encoding FOXP3.

In such an embodiment, the CAR may comprise:
(a) a leader sequence comprising or consisting of a sequence as set out in SEQ ID NO. 42, or a sequence having at least 80% sequence identity thereto;
(b) an antigen binding domain comprising or consisting of a sequence as set out in SEQ ID NO. 33, 34, 35 or 36, or a sequence having at least 80% sequence identity thereto;
(c) a CD8α hinge and transmembrane domain sequence comprising or consisting of the sequence as set forth in SEQ ID NO. 43, or a sequence having at least 80% sequence identity thereto;
(d) a C28 co-stimulatory domain comprising or consisting of the sequence as set forth in SEQ ID NO. 44, or a sequence having at least 80% sequence identity thereto;
(e) a CD3ζ signalling domain comprising or consisting of the sequence as set forth in SEQ ID NO. 45, or a sequence having at least 80% sequence identity thereto.

As is clear from the above description in addition to the specific polypeptide and nucleotide sequences mentioned herein, also encompassed is the use of variants, or derivatives and fragments thereof.

The term “derivative” or “variant” as used interchangeably herein, in relation to proteins or polypeptides of the present invention includes any substitution of, variation of, modification of, replacement of, deletion of and/or addition of one (or more) amino acid residues from or to the sequence providing that the resultant protein or polypeptide retains the desired function (for example, where the derivative or variant is an antigen binding domain, the desired function may be the ability of the antigen binding domain to bind its target antigen (for example, a variant of an antigen binding domain which binds to TREM2 retains the ability to bind TREM2), where the derivative or variant is a signalling domain, the desired function may be the ability of that domain to signal (e.g. activate or inactivate a downstream molecule), where the derivative or variant is a transcription factor (e.g. FOXP3), the desired function may be the ability of the transcription factor to bind to target DNA and/or to induce transcription or where the derivative or variant is a safety switch polypeptide, the desired function may be the ability of that polypeptide to induce cell death e.g. upon binding of a molecule thereto. Alternatively viewed, the variants or derivatives referred to herein are functional variants or derivatives. For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding reference sequence or may have an increased level of function (e.g. increased by at least 10%, at least 20%, at least 30%, at least 40% or at least 50%). For example, a variant antigen recognition domain of a CAR of the invention may have at least 10, 20, 30, 40, 50, 60, 70, 80 or 90% of the binding affinity for TREM2 as the reference antigen recognition domain.

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 1 below.

TABLE 1

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The derivative may be a homologue. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 70%, 75%, 85% or 90% identical, preferably at least 95%, 96%, 97%, 98% or 99% identical to the subject sequence. Typically, the variants will comprise the same active sites etc. as the subject amino acid sequence. Although homology can also be considered in terms of similarity (i.e. amino acid residues having similar chemical properties/functions), in the context herein it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence.

Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally, e.g. is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Nucleic acid molecules and polynucleotides/nucleic acid sequences as defined herein may comprise DNA or RNA. They may be single-stranded or double-stranded. It will be understood by a skilled person that numerous different nucleic acid molecules/polynucleotides can encode the same polypeptide as a result of the degeneracy of the genetic code. In addition, it is to be understood that the skilled person may, using routine techniques, make nucleotide substitutions that do not affect the polypeptide sequence encoded by the nucleic acid molecules/polynucleotides/nucleotide sequences as defined herein to reflect the codon usage of any particular host organism in which the polypeptides of the invention are to be expressed.

The nucleic acid molecules/polynucleotides may be modified by any method available in the art. Such modifications may be carried out in order to enhance the in vivo activity or lifespan of the nucleic acid molecules/polynucleotides as defined herein.

Nucleic acid molecules/polynucleotides/nucleotide sequences such as DNA nucleic acid molecules/polynucleotides/sequences may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The present nucleic acid molecule/polynucleotide may further comprise a nucleic acid sequence encoding a selectable marker. Suitably selectable markers are well known in the art and include, but are not limited to, fluorescent proteins—such as GFP. Suitably, the selectable marker may be a fluorescent protein, for example GFP, YFP, RFP, tdTomato, dsRed, or variants thereof. In some embodiments the fluorescent protein is GFP or a GFP variant. The nucleic acid sequence encoding a selectable marker may be provided in combination with a nucleic acid molecule herein in the form of a nucleic acid construct. Such a nucleic acid construct may be provided in a vector.

Suitably, the selectable marker/reporter domain may be a luciferase-based reporter, a PET reporter (e.g. Sodium Iodide Symporter (NIS)), or a membrane protein (e.g. CD34, low-affinity nerve growth factor receptor (LNGFR)).

The nucleic acid sequences encoding one or more selectable markers may be separated from the present nucleic acid molecule, and/or from each other, by one or more co-expression sites which enables expression of each polypeptide as a discrete entity. Suitable co-expression sites are known in the art and include, for example, internal ribosome entry sites (IRES) and self-cleaving sites such as those included in the present nucleic acid molecules, and as defined above. In an embodiment this may be a 2A cleavage sites, as discussed above.

The use of a selectable marker is advantageous as it allows cells (e.g., Tregs) in which a nucleic acid molecule, construct or vector of the present invention has been successfully introduced (such that the encoded TREM2 CAR and other modules, e.g., FOXP3 and safety switch polypeptide, are expressed) to be selected and isolated from a starting cell population using common methods, e.g., flow cytometry.

The nucleic acid molecule/polynucleotides used in the present invention may be codon-optimised. Codon optimisation has previously been described in WO1999/41397 and WO2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

The constructs of the present invention may comprise one or more regulatory sequences, for example a promoter. A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person. The promoter may be from any source, and may be a viral promoter, or a eukaryotic promoter, including mammalian or human promoters (i.e. a physiological promoter). In an embodiment the promoter is a viral promoter. Particular promoters include LTR promoters, EFS (or functional truncations thereof), SFFV, PGK, and CMV. In an embodiment the promoter is SFFV or a viral LTR promoter. Particularly, a SFFV promoter may be used within a nucleic acid molecule, construct or vector of the invention to allow initiation of transcription of the nucleotide sequence(s). The promoter may thus control the expression of the CAR of the invention. Where there is more than one nucleotide sequence, each sequence may be operably linked to the same promoter, e.g. nucleotide sequences encoding the CAR, FOXP3 and/or the safety switch.

The SFFV promoter may comprise a nucleotide sequence as set out in SEQ ID NO. 67.

“Operably linked to the same promoter” means that transcription of the polynucleotide sequences may be initiated from the same promoter (e.g., transcription of the first, second and third polynucleotide sequences is initiated from the same promoter) and that the nucleotide sequences are positioned and oriented for transcription to be initiated from the promoter. Polynucleotides operably linked to a promoter are under transcriptional regulation of the promoter.

In some embodiments of the invention, the polynucleotide is within an expression vector. The term “expression vector” as used herein means a construct enabling expression of the CAR polypeptide and any additional polypeptides such as a FOXP3 polypeptide or safety switch polypeptide.

A vector is a tool that allows or facilitates the transfer of an entity from one environment to another. As used herein, and by way of example, some vectors used in recombinant nucleic acid techniques allow entities, such as a segment of nucleic acid (e.g., a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g., in vitro transcribed mRNAs), chromosomes, artificial chromosomes and viruses. The vector may also be, for example, a naked nucleic acid (e.g., DNA). In its simplest form, the vector may itself be a nucleotide of interest.

The vectors used herein may be, for example, plasmid, mRNA or virus vectors and may include a promoter (as described above) for the expression of a nucleic acid molecule/polynucleotide and optionally a regulator of the promoter.

In an embodiment the vector is a viral vector, for example a retroviral, e.g., a lentiviral vector or a gamma retroviral vector.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal.

Suitably, the vector may include a 5′LTR and a 3′LTR.

The vector may comprise one or more additional regulatory sequences which may act pre- or post-transcriptionally. “Regulatory sequences” are any sequences which facilitate expression of the polypeptides, e.g., act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g., operably linked to the promoter.

Vectors comprising the present nucleic acid molecules/polynucleotides may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a nucleic acid molecule or construct.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Although the present nucleic acid molecules are designed to be used as single constructs, and this would be contained in a single vector, it is not precluded that they are introduced into a cell in conjunction with other vectors, for example encoding other polypeptides it may be desired also to introduce into the cell.

Engineered cells may be generated by introducing a nucleic acid molecule, construct, or vector as defined herein, by one of many means including transduction with a viral vector, and transfection with DNA or RNA.

The present cell may be made by: introducing to a cell (e.g. by transduction or transfection) the nucleic acid molecule/polynucleotide, construct or vector as defined herein.

Suitable cells are discussed further below, but the cell may be from a sample isolated from a subject. The subject may be a donor subject, or a subject for therapy (i.e., the cell may be an autologous cell, or a donor cell, for introduction to another recipient, e.g., an allogeneic cell).

The cell may be generated by a method comprising the following steps:
(i) isolation of a cell-containing sample from a subject or provision of a cell-containing sample; and
(ii) introduction into (e.g., by transduction or transfection) the cell-containing sample of a nucleic acid molecule, construct, or vector as defined herein, to provide a population of engineered cells.

A target cell-enriched sample may be isolated from, enriched, and/or generated from the cell-containing sample prior to and/or after step (ii) of the method. For example, isolation, enrichment and/or generation of Tregs (or other target cells) may be performed prior to and/or after step (ii) to isolate, enrich or generate a Treg-enriched sample. Isolation and/or enrichment from a cell-containing sample may be performed after step (ii) to enrich for cells and/or Tregs (or other target cells) comprising the CAR, the nucleic acid molecule/polynucleotide, the construct and/or the vector as described herein.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example, from Tcon cells by introducing DNA or RNA coding for FOXP3 and/or from ex-vivo differentiation of inducible progenitor cells or embryonic progenitor cells. Methods for isolating and/or enriching other target cells are known in the art.

Suitably, an engineered target cell may be generated by a method comprising the following steps:
(i) isolation of a target-cell enriched sample from a subject or provision of a target cell-enriched sample; and
(ii) introduction into (e.g., by transduction or transfection) the target cell-enriched sample of a nucleic acid, construct or vector as defined herein, to provide a population of engineered target cells.

The target cell may be a Treg cell, or precursor or a progenitor thereof.

An “engineered cell” means a cell which has been modified to comprise or express a polynucleotide which is not naturally encoded by the cell. Methods for engineering cells are known in the art and include, but are not limited to, genetic modification of cells e.g., by transduction such as retroviral or lentiviral transduction, transfection (such as transient transfection—DNA or RNA based) including lipofection, polyethylene glycol, calcium phosphate and electroporation, as discussed above. Any suitable method may be used to introduce a nucleic acid sequence into a cell. Non-viral technologies such as amphipathic cell penetrating peptides may be used to introduce nucleic acid.

Accordingly, the nucleic acid molecule as described herein is not naturally expressed by a corresponding, unmodified cell. Indeed, the nucleic acid molecule encoding the CAR is an artificial construct, and in an embodiment the safety switch polypeptide is an artificial construct, such they could not occur or be expressed naturally. Suitably, an engineered cell is a cell which has been modified e.g., by transduction or by transfection. Suitably, an engineered cell is a cell which has been modified or whose genome has been modified e.g., by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign nucleic acid, e.g., DNA or RNA, into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells may be generated by introducing a nucleic acid as described herein by one of many means including transduction with a viral vector, transfection with DNA or RNA. Cells may be activated and/or expanded prior to, or after, the introduction of a nucleic acid as described herein, for example by treatment with an anti-CD3 monoclonal antibody or both anti-CD3 and anti-CD28 monoclonal antibodies. The cells may also be expanded in the presence of anti-CD3 and anti-CD28 monoclonal antibodies in combination with IL-2. Suitably, IL-2 may be substituted with IL-15. Other components which may be used in a cell (e.g., Treg) expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The cell may be an immune cell, or a precursor therefor. A precursor cell may be a progenitor cell. Representative immune cells thus include T-cells, in particular, cytotoxic T-cells (CTLs; CD8+ T-cells), helper T-cells (HTLs; CD4+ T-cells) and regulatory T cells (Tregs). Other populations of T-cells are also useful herein, for example naive T-cells and memory T-cells. Other immune cells include NK cells, NKT cells, dendritic cells, MDSC, neutrophils, and macrophages. Precursors of immune cells include pluripotent stem cells, e.g., induced PSC (iPSC), or more committed progenitors including multipotent stem cells, or cells which are committed to a lineage. Precursor cells can be induced to differentiate into immune cells in vivo or in vitro. In one aspect, a precursor cell may be a somatic cell which is capable of being transdifferentiated to an immune cell of interest.

Most notably, the immune cell may be an NK cell, a dendritic cell, a MDSC, or a T cell, such as a cytotoxic T lymphocyte (CTL), helper T cell or a Treg cell.

In a preferred embodiment the immune cell is a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

A T cell as used herein is a lymphocyte including any type of T cell, such as an alpha beta T cell (e.g., CD8 or CD4+), a gamma delta T cell, a memory T cell, a Treg cell.

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs as disclosed herein may proliferate 5%, 10%, 20%, 30%, 40%, 50%, 60%, 70%, 90%, 95% or 99% less than the same Tconv cells cultured in the absence of the Tregs. For example, antigen-specific Tconv cells co-cultured with the present Tregs may proliferate 5%, 10%, 20%, 30%, 40%, 50%, 60%, 70%, 90%, 95% or 99% less than the same Tconv cells cultured in the presence of non-engineered Tregs. The cells comprising the nucleic acid, expression construct or vector as defined herein, e.g., Tregs, may have an increased suppressive activity as compared to non-engineered Tregs (e.g., an increased suppressive activity of at least 5, 10, 20, 30, 40, 50, 60, 70, 80 or 90%).

Antigen-specific Tconv cells co-cultured with the Tregs herein may express at least 10%, at least 20%, at least 30%, at least 40%, at least 50%, or at least 60% less effector cytokine than corresponding Tconv cells cultured in the absence of the Tregs (e.g., in the presence of non-engineered Tregs). The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4⁺CD25⁺FOXP3⁺).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7(10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

Suitably, the Treg may be a natural Treg (nTreg). As used herein, the term “natural T reg” means a thymus-derived Treg. Natural Tregs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38(6), pp. 1654-1663).

Further suitable Tregs include, but are not limited to, Tr cells (which do not express Foxp3, and have high IL-10 production); CD8⁺FOXP3⁺ T cells; and γδ FOXP3⁺ T cells.

Different subpopulations of Tregs are known to exist, including naïve Tregs (CD45RA⁺FoxP3^{low}), effector/memory Tregs (CD45RA-FoxP3^{high}) and cytokine-producing Tregs (CD45RA-FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g., less than 50, 60, 70, 80 or 90% suppressive than naïve Tregs). Reference to expression levels herein may refer to mRNA or protein expression. Particularly, for cell surface markers such as CD45RA, CD25, CD4, CD45RO etc, expression may refer to cell surface expression, i.e., the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA⁺. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g., a memory Treg cell). Alternatively viewed, a naïve Treg cell may express at least 2, 3, 4, 5, 10, 50 or 100-fold the amount of CD45RA as compared to a non-naïve Treg cell (e.g., a memory Treg cell). The level of expression of CD45RA can be readily determined by methods of the art, e.g., by flow cytometry using commercially available antibodies. Typically, non-naïve Treg cells do not express CD45RA or low levels of CD45RA.

Particularly, naïve Tregs may not express CD45RO, and may be considered to be CD45RO⁻. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor.

Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g., at least 10, 20, 30, 40, 50, 60, 70, 80 or 90% lower level of expression. Naïve Tregs may further express additional markers, including CCR7⁺ and CD31⁺.

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out, and methods for determining the presence and/or levels of expression of cell markers are well-known in the art and include, for example, flow cytometry, using commercially available antibodies.

Suitably, the cell, such as a Treg, is isolated from peripheral blood mononuclear cells (PBMCs) obtained from a subject. Suitably the subject from whom the PBMCs are obtained is a mammal, preferably a human. Suitably the cell is matched (e.g. HLA matched) or is autologous to the subject to whom the engineered cell is to be administered. Suitably, the subject to be treated is a mammal, preferably a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered cell is to be administered.

Suitably, the Treg is part of a population of cells. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells (e.g. iPSCs) or embryonic progenitor cells to the Treg. A nucleic acid molecule or vector as described herein may be introduced into the inducible progenitor cells or embryonic progenitor cells prior to, or after, differentiation to a Treg. Suitable methods for differentiation are known in the art and include that disclosed in Haque et al, J Vis Exp., 2016, 117, 54720 (incorporated herein by reference).

As used herein, the term “conventional T cell” or Tcon or Tconv (used interchangeably herein) means a T lymphocyte cell which expresses an as T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may be generated from a Tcon by introducing the nucleic acid which includes a sequence coding for FOXP3. Alternatively, the engineered Treg may be generated from a Tcon by in vitro culture of CD4+CD25−FOXP3− cells in the presence of IL-2 and TGF-β.

In another embodiment the target cell into which the nucleic acid molecule, construct or vector is introduced is not a cell intended for therapy. In an embodiment the cell is a production host cell. The cell may be for production of the nucleic acid, e.g., cloning, or vector, or polypeptides.

The invention also provides a cell population comprising a cell as defined or described herein. It will be appreciated that a cell population may comprise both cells of the invention comprising a nucleic acid molecule, expression construct or vector as defined herein, and cells which do not comprise a nucleic acid molecule, expression construct or vector of the invention, e.g., untransduced or untransfected cells. Although in a preferred embodiment, all the cells in a population may comprise a nucleic acid, expression construct or vector of the invention, cell populations having at least 10, 20, 30, 40, 50, 60, 70, 80, 90, 95 or 99% of cells comprising a nucleic acid, expression construct or vector of the invention are provided.

There is also provided a pharmaceutical composition comprising a cell or cell population as defined or described herein, a vector as defined herein. The vector may be used for gene therapy. Thus, rather than administering a cell, a vector may be administered instead, to modify endogenous cells in the subject to express the introduced nucleic acid molecule. Vectors suitable for use in gene therapy are known in the art, and include viral vectors.

Thus, in a further aspect, the invention provides a cell, cell population or pharmaceutical composition as defined herein for use in therapy.

A pharmaceutical composition is a composition that comprises or consists of a therapeutically effective amount of a pharmaceutically active agent i.e., the cell (e.g., Treg), cell population or vector. It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” it is included that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The cells, cell population or pharmaceutical compositions may be administered in a manner appropriate for treating and/or preventing the desired disease or condition. The quantity and frequency of administration will be determined by such factors as the condition of the subject, and the type and severity of the subject's disease or condition, although appropriate dosages may be determined by clinical trials. The pharmaceutical composition may be formulated accordingly.

The cell, cell population or pharmaceutical composition as described herein can be administered parenterally, for example, intravenously or intrathecally, or they may be administered by infusion techniques. In one embodiment, the cell, cell population or pharmaceutical composition as described herein is administered intrathecally (e.g., via intra-cisterna magna (ICM) or lumbar puncture (LP) delivery). The cell, cell population or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise cells in infusion media, for example sterile isotonic solution. The pharmaceutical composition may be enclosed in ampoules, disposable syringes or multiple dose vials made of glass or plastic.

The cell, cell population or pharmaceutical composition may be administered in a single or in multiple doses. Particularly, the cell, cell population or pharmaceutical composition may be administered in a single, one-off dose. The pharmaceutical composition may be formulated accordingly.

Depending upon the disease/condition and subject to be treated, as well as the route of administration, the cell, cell population or pharmaceutical composition may be administered at a specific stage of disease. An optimum time to administer the cell, cell population or pharmaceutical composition of the invention may be at a point in disease where the level of TREM2 expression is high enough for the cell, cell population or pharmaceutical composition to traffic to the disease site but where damage to the motor neurons may not be so severe that the cell, cell population or pharmaceutical composition is unable to have a therapeutic effect. In one embodiment, the cell, cell population or pharmaceutical composition of the invention may be administered when neuronal expression levels of TREM2 are at least 30, 40, 50, 60 or 70% higher than in patients without ALS or than in the same patient at the beginning of disease (e.g., when the patient has a ALSFRS-R score of 48, and/or normal physical function). TREM2 expression levels can be assessed by methods known in the art, for example by assessing mRNA levels using conventional techniques (e.g., Northern blotting). The revised Amyotrophic Lateral Sclerosis Functional Rating Scale (ALSFRS-R) is a rating sale for monitoring the progression of disability in patients with ALS and thus correlates with disease severity. An ALSFRS-R score of 48 indicates normal physical function (and thus mild disease) and a score of 0 indicates severe physical impairment (and thus severe disease). Therefore, in one embodiment, the cell, cell population or pharmaceutical composition of the invention may be administered to a subject with a low, medium or high ALSFRS-R score, i.e., with low, medium or high disease severity.

Alternatively viewed, the cell, cell population or pharmaceutical composition of the invention may be administered to a subject with an ALSFRS-R score of between about 48 to 30, 30 to 20, or 20 to 0, i.e., with an ALSFRS-R score of 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 or 0.

The pharmaceutical composition may further comprise one or more active agents. The pharmaceutical composition may further comprise one or more other therapeutic agents, such as lympho-depletive agents (e.g., thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL41g), and/or drugs inhibiting specific cytokines (IL-6, IL-17, TNFalpha, IL18).

Depending upon the disease/condition and subject to be treated, as well as the route of administration, the cell, cell population or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for the cells herein, doses of 5×10⁷ to 3×10⁹ cells, or 10⁸ to 2×10⁹ cells per subject may be administered.

The cell may be appropriately modified for use in a pharmaceutical composition. For example, cells may be cryopreserved and thawed at an appropriate time, before being infused into a subject.

The invention further includes the use of kits comprising the cell, cell population and/or pharmaceutical composition herein. Preferably said kits are for use in the methods and uses as described herein, e.g., the therapeutic methods as described herein. Preferably said kits comprise instructions for use of the kit components.

The cell, cell population and pharmaceutical composition of the invention may find particular utility in the treatment of disorders associated with cells that express TREM2 or release soluble TREM2, or with disorders where soluble TREM2 is localised at or near the site of disease, particularly disorders that would benefit from the immunosuppressive activity or target killing activity of the cells of the invention.

The cells, cell populations, compositions and vectors herein may be for use in treating, preventing or reducing the risk of a disease or condition in a subject, notably a disease or condition which may be treated by or with the CAR. The cells and compositions containing them are for adoptive cell therapy (ACT). Various conditions may be treated by administration of cells, including particularly Treg cells, expressing a CAR according to the present disclosure. As noted above, this may be conditions responsive to immunosuppression, and particularly the immunosuppressive effects of Tregs cells. The cells, cell populations, compositions and vectors described herein may thus be used for inducing, or achieving, immunosuppression in a subject. The Treg cells administered, or modified in vivo, may be targeted by expression of the CAR. Conditions suitable for such treatment include neurological diseases or disorders, such as neuronal damage, neuroinflammation or neurodegeneration, and autoimmune or inflammatory diseases (e.g., type I diabetes), or more broadly a condition associated with any undesired or unwanted or deleterious immune response. Additionally, the cells, cell populations, compositions and vectors herein may be for use in promoting tissue repair and/or tissue regeneration.

Conditions to be treated or prevented include inflammation, or alternatively put, a condition associated with or involving inflammation. Inflammation may be chronic or acute.

Furthermore, the inflammation may be low-level or systemic inflammation. For example, the inflammation may be inflammation which occurs in the context of neurodegeneration, or neuronal damage/injury.

An inflammatory disorder is any condition associated with unwanted inflammation or with an increase in inflammation. Inflammatory disorders include conditions such as inflammatory bowel disease. The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis and juvenile onset diabetes.

The term “target cell” refers to any cell expressing TREM2 to which the cell of the invention is to be directed to exert its therapeutic effect. In some embodiments, the target cell functions as a marker of a disease site, i.e. to attract the cells of the invention to provide an immunosuppressive effect. In some embodiments, the target cell is killed or abrogated by the cells of the invention. As noted above, in some embodiments, the target cell will be a microglial cell. A skilled person will appreciate that TREM2 may also be cleaved and the extracellular domain secreted from a cell, and therefore that the cells of the invention may be directed to secreted proteins which are not present within or on a cell.

In some embodiments, the disease or disorder to be treated is a neurological disease or disorder/condition. In some embodiments, the neurological disease or disorder is associated with inflammation. Thus, in some embodiments, the invention may find utility in treating or preventing (e.g. reducing the risk of) neuroinflammation or an associated disease or disorder. The neuroinflammation may be chronic or acute, preferably chronic. The neuroinflammation may be neuroinflammation of the central or peripheral nervous system, preferably the central nervous system.

The engineered cells, e.g., Tregs, may be administered to a subject with a disease in order to lessen, reduce, or improve at least one symptom of disease such as muscle weakness, muscle twitches, stiff muscles, muscle wasting, cognitive decline, dementia, behavioural changes, pain and/or fatigue. The at least one symptom may be lessened, reduced, or improved by at least 10%, at least 20%, at least 30%, at least 40%, or at least 50%, or the at least one symptom may be completely alleviated.

The engineered cells, e.g., Tregs may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells are not administered, or progression of the disease may be completely stopped.

Suitably, where the subject to be treated is suffering from a neurological disease, disorder or injury, the neurological disease, disorder or injury may be selected from amyotrophic lateral sclerosis (ALS), frontotemporal dementia (FTD), progressive supranuclear palsy (PSP), Parkinson's disease, Alzheimer's disease, Huntington's disease, multiple sclerosis, vascular dementia, mixed dementia, Creutzfeldt-Jakob disease, Chronic Inflammatory Demyelinating Polyneuropathy (CIDP), Taupathy disease, Nasu-Hakola disease, central nervous system lupus, dementia with Lewy bodies, Multiple System Atrophy (Shy-Drager syndrome), progressive supranuclear palsy, cortical basal ganglionic degeneration, acute disseminated encephalomyelitis, seizures, spinal cord injury, traumatic brain injury (e.g. ischemia and traumatic brain injury), depression and autism spectrum disorder. In particular, the neurological disease, disorder or injury may be selected from amyotrophic lateral sclerosis (ALS), frontotemporal dementia (FTD), progressive supranuclear palsy (PSP), Parkinson's disease, Alzheimer's disease, Huntington's disease or multiple sclerosis. As mentioned above, CAR-Tregs specific for TREM2 may be able to traffic to sites of TREM2 expression and control inflammation, via their bystander effect, to slow down the rate of neurodegeneration in these diseases.

In one embodiment, the subject may have ALS.

Suitably, the subject is a mammal. Suitably, the subject is a human. “ALS” or amyotrophic lateral sclerosis (also known as motor neuron disease or Lou Gehrig's disease) as described herein is a progressive neurodegenerative disorder involving primarily motor neurons in the cerebral cortex, brainstem and spinal cord. It is a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

There are several ALS and ALS-like syndromes, all of which may be treated by the cells, cell populations or pharmaceutical compositions of the present invention. These include, for example, sporadic ALS, genetically-determined (familial, hereditary) ALS, Primary Lateral Sclerosis (PLS), Progressive Muscular Atrophy (PMA), ALS-Plus syndromes, ALS with Laboratory Abnormalities of Uncertain Significance, ALS-mimic syndromes (including post-poliomyelitis syndrome, multifocal motor neuropathy with or without conduction block, endocrinopathies, especially hyperparathyroid or hyperthyroid states, lead intoxication, infection and paraneoplastic syndromes) (Brooks et al., 2000, ALS and other motor neuron disorders, 1, 293-299). The cell, cell population or pharmaceutical composition of the invention may be for use in treating or preventing sporadic ALS, familial ALS, PLS, PMA, ALS-Plus syndromes, ALS with Laboratory Abnormalities of Uncertain Significance, or ALS-mimic syndromes. Preferably, the cell, cell population or pharmaceutical composition of the present invention may be for use in treating or preventing sporadic or familial ALS.

ALS can also be classified in several ways: by whether it is familial or sporadic, as mentioned above; by the types of motor neuron affected; by the region first affected; and by how fast the disease progresses, which is related to the age of onset. Again, the cells, cell populations or pharmaceutical compositions of the present invention may be for use in treating or preventing any form of ALS, however it is classified. It should be noted that the classifications described herein may overlap and therefore where it is stated that the cell, cell population or pharmaceutical composition of the invention can treat or prevent a particular classification of ALS, this is not to the exclusion of other classifications. For example, a subject with classic ALS may also have young-onset ALS; therefore, the cell, cell population or pharmaceutical composition of the present invention may be for use in treating or preventing classic ALS and young onset ALS.

Typical or “classical” ALS involves upper motor neurons in the brain, and lower motor neurons in the spinal cord. Primary Lateral Sclerosis (PLS) involves only upper motor neurons, and Progressive Muscular Atrophy (PMA) involves only lower motor neurons.

Classic ALS accounts for about 70% of all cases of ALS and can be subdivided into limb-onset ALS (also known as spinal-onset) and bulbar-onset ALS. Limb-onset ALS begins with weakness in the arms and legs and accounts for about two-thirds of all classic ALS cases. Bulbar-onset ALS begins with weakness in the muscles of speech, chewing, and swallowing and accounts for the other one-third of cases. Bulbar onset is associated with a worse prognosis than limb-onset ALS. A rare variant is respiratory-onset ALS that accounts for about 3% of all cases of ALS, in which the initial symptoms are difficulty breathing (dyspnea) with exertion, at rest, or while lying down (orthopnea). Respiratory-onset ALS has the worst prognosis of any ALS variant, with a median survival of 1.4 years (Chio et al., 2011, Journal of Neurology, Neurosurgery, and Psychiatry, 82 (7): 740-46). The cell, cell population or pharmaceutical composition of the invention may be for use in treating or preventing classic ALS, PLS or PMA. More specifically, the cell, cell population or pharmaceutical composition of the invention may be for use in treating or preventing limb-onset ALS, bulbar-onset ALS, respiratory-onset ALS, PLS or PMA.

Regional variants of ALS have symptoms that are limited to a single spinal cord region for at least a year; they progress more slowly than classic ALS and are associated with longer survival. Examples include flail arm syndrome, flail leg syndrome, and isolated bulbar ALS. The cell, cell population or pharmaceutical composition of the invention may be for use in treating or preventing flail arm syndrome, flail leg syndrome, or isolated bulbar ALS.

ALS can also be classified based on the age of onset. Most patients with sporadic ALS develop the disease between the ages of 58 to 63 and most patients with familial ALS develop the disease between the ages of 47 to 52 and this is known as adult-onset ALS. However, 10% of all cases of ALS begin before age 45, known as young-onset ALS, and about 1% of all cases begin before age 25, known as juvenile ALS. Cases in patients over the age of 70 are known as late-onset ALS. The cell, cell population or pharmaceutical composition of the invention may be for use in treating or preventing juvenile ALS, young-onset ALS, adult-onset ALS or late-onset ALS.

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include frontotemporal dementia, Alzheimer's disease, vascular dementia, mixed dementia, semantic dementia, and dementia with Lewy bodies.

“FTD” or frontotemporal dementia (also known as frontotemporal degeneration disease or frontotemporal neurocognitive disorder) as described herein encompasses several types of dementia involving the deterioration of the frontal and temporal lobes. These include a behavioural variant (bvTFD) previously known as Pick's disease, two variants of primary progressive aphasia (semantic variant (svPPA) and nonfluent variant (nfvPPA)) and other related disorders such as FTD with ALS (also known as FTD-ALS or FTD-MND). FTD accounts for 20% of pre-senile dementia cases. A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain (e.g. intraneuronal accumulations of hyperphosphorylated Tau protein in neurofibrillary tangles or Pick bodies). The cell, cell population or pharmaceutical composition of the present invention may be for use in treating or preventing FTD.

“PSP” or progressive supranuclear palsy (PSP) is an uncommon brain disorder that results from damage to nerve cells in the brain. PSP affects movement, control of walking (gait), balance, speech, swallowing, vision, mood, behavior, thinking, and control of eye movements. The symptoms of PSP are caused by a gradual deterioration of brain cells in a few specific areas in the brain, mainly in the region called the brain stem. PSP is characterized by abnormal deposits of the protein tau in nerve cells in the brain.

There are various types of PSP, including classical Richardson syndrome (PSP-RS), PSP-Parkinsonism (PSP-P), PSP-pure akinesia with gait freezing (PSP-PAGF), frontal PSP, PSP-corticobasal syndrome (PSP-CBS), PSP-behavioural variant of frontotemporal dementia (PSP-bvFTD), PSP-progressive non-fluent aphasia (PSP-PNFA), PSP-C and PSP induced by Annonaceae. The cell, cell population or pharmaceutical composition of the present invention may be for use in treating or preventing PSP.

“Parkinson's disease”, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems. The cell, cell population or pharmaceutical composition of the present invention may be for use in treating or preventing Parkinson's disease.

“Alzheimer's disease” (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

1-2% of Alzheimer's cases are inherited, known as early onset familial Alzheimer's disease, but most cases are not inherited and are termed sporadic Alzheimer's disease. The cell, cell population or pharmaceutical composition of the present invention may be for use in treating or preventing Alzheimer's disease.

“Huntington's disease” (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior. The cell, cell population or pharmaceutical composition of the present invention may be for use in treating or preventing Huntington's disease.

Tauopathy diseases, or “Tauopathies”, are a class of neurodegenerative disease caused by aggregation of the microtubule-associated protein tau within the brain. Alzheimer's disease (AD) is the most well-known Taupathy disease, and involves an accumulation of tau protein within neurons in the form of insoluble neurofibrillary tangles (NFTs). Other Taupathy diseases and disorders include progressive supranuclear palsy, dementia pugilistica (chromic traumatic encephalopathy), Frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, frontotemporal dementia, and frontotemporal lobar degeneration. The cell, cell population or pharmaceutical composition of the present invention may be for use in treating or preventing Tauopathies.

“Multiple sclerosis” (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

Four disease courses have been identified in multiple sclerosis: clinically isolated syndrome (CIS), relapsing-remitting MS (RRMS), primary progressive MS (PPMS), and secondary progressive MS (SPMS). The cell, cell population or pharmaceutical composition of the present invention may be for use in treating or preventing Multiple sclerosis. More particularly, the cell, cell population or pharmaceutical composition of the present invention may be for use in treating or preventing clinically isolated syndrome (CIS), relapsing-remitting MS (RRMS), primary progressive MS (PPMS), or secondary progressive MS (SPMS).

Creutzfeldt-Jakob disease (CJD) is a prion disease which has sporadic, iatrogenic, and familial forms. CJD is characterized by spongiform change (e.g., microcavitation of the brain, usually predominant in gray matter), neuronal cell loss, astrocytic proliferation disproportionate to neuronal loss, and accumulation of an abnormal amyloidogenic protein, sometimes in discrete plaques in the brain. Prions, the infectious agents that transmit these diseases differ markedly from viruses and viroids in that no chemical or physical evidence for a nucleic acid component has been reproducibly detected in infectious materials. The cell, cell population or pharmaceutical composition of the present invention may be for use in treating or preventing CJD.

Central nervous system (CNS) lupus is a neurologic manifestation of systemic lupus erythematosus (SLE), a multisystem autoimmune connective tissue disorder. CNS lupus is a serious illness with neurologic symptoms which include headaches, confusion, fatigue, depression, seizures, strokes, vision problems, mood swings, and difficulty concentrating. The cell, cell population or pharmaceutical composition of the present invention may be for use in treating or preventing CNS lupus.

Multiple system atrophy (MSA), also known as Shy-Drager syndrome, is a progressive neurodegenerative disorder characterized symptoms that affect both the autonomic nervous system and movement. Symptoms are the result of progressive loss of function and death of different types of nerve cells in the brain and spinal cord and include fainting spells, heart rate problems, and bladder control. Motor impairments include tremor, rigidity, loss of muscle coordination, and difficulties with speech and gait. MSA includes disorders that historically had been referred to as Shy-Drager syndrome, olivopontocerebellar atrophy, and striatonigral degeneration. A distinguishing feature of MSA is the accumulation of the protein alpha-synuclein in glia, the cells that support nerve cells in the brain. The cell, cell population or pharmaceutical composition of the present invention may be for use in treating or preventing MSA.

Cortical basal ganglionic degeneration (CBGD) is a rare, progressive neurodegenerative disease involving the cerebral cortex and the basal ganglia. CBGD symptoms include movement and cognitive dysfunction, Parkinsonism, alient hand syndrome, and psychiatric disorders. CBGD pathology is characterized by the presence of astrocytic abnormalities within the brain and improper accumulation of the protein tau. The cell, cell population or pharmaceutical composition of the present invention may be for use in treating or preventing CBGD.

Acute disseminated encephalomyelitis (ADEM), or acute demyelinating encephalomyelitis, is a rare autoimmune disease characterized by widespread inflammation in the brain and spinal cord. ADEM also damages myelin insulation on nerves of the CNS, destroying the white matter. ADEM is characterized by multiple inflammatory lesions in the subcortical and central white matter and cortical gray-white junction of the cerebral hemispheres, cerebellum, brainstem, and spinal cord. The cell, cell population or pharmaceutical composition of the present invention may be for use in treating or preventing ADEM. Nasu-Hakola disease (NHD), which may alternatively be referred to as polycystic lipomembranous osteodysplasia with sclerosing leukoencephalopathy (PLOSL), is a rare inherited leukodystrophy characterized by progressive presenile dementia associated with recurrent bone fractures due to polycystic osseous lesions of the lower and upper extremities. NHD disease course is generally divided into four stages: latent, osseous, early neurologic, and late neurologic. After a normal development during childhood (latent stage), NHD starts manifesting during adolescence or young adulthood (typical age of onset 20-30 years) with pain in the hands, wrists, ankles, and feet. Patients then start suffering from recurrent bone fractures due to polycystic osseous and osteroporotic lesions in the limb bones (osseous stage). During the third or fourth decade of life (early neurologic stage), patients present with pronounced personality changes (e.g., euphoria, lack of concentration, loss of judgment, and social inhibitions) characteristic of a frontal lobe syndrome. Patients also typically suffer from progressive memory disturbances. Epileptic seizures are also frequently observed. Finally (late neurologic stage), patients progress to a profound dementia, are unable to speak and move, and usually die by the age of 50. The cell, cell population or pharmaceutical composition of the present invention may be for use in treating or preventing NHD.

Neurological injuries can result from stroke, acute trauma, chronic trauma, seizures, spinal cord injury, traumatic brain injury (TBI), alcohol abuse, or vitamin B deficiency. Neurological injuries can result in impairment or disability, including neurocognitive deficits, delusions, speech or movement problems, intellectual disability, sleep disorders, mental fatigue, personality changes, coma or a persistent vegetative state. The cell, cell population or pharmaceutical composition of the present invention may be for use in treating or preventing neurological injuries.

TREM2 is expressed in macrophages that are associated with various diseases and/or disease sites, such as fibrosis (e.g., renal fibrosis) and atherosclerosis plaques. Thus, the disease or disorder to be treated may be a disease or disorder/condition associated with macrophages expressing TREM2. Microglia are a specialised population of macrophages that are found in the CNS. Thus, the disease or disorder to be treated may be a disease or disorder/condition associated with microglia expressing TREM2.

Suitably, the cell may be an engineered Treg cell and the cell population may be a population of engineered Treg cells, which have been engineered to express a CAR as described herein.

Suitably, the CAR may comprise an antigen binding domain which is capable of specifically binding to TREM2, i.e., the antigen is TREM2.

A method for treating a disease or condition relates to the therapeutic use of the cells herein. In this respect, the cells may be administered to a subject having an existing disease or condition in order to lessen, reduce or improve at least one symptom associated with the disease or condition and/or to slow down, reduce or block the progression of the disease.

Suitably, treating and/or preventing a neurodegenerative, autoimmune or inflammatory disease may refer to administering an effective amount of the cells (e.g., Tregs) such that the amount of existing medication that a subject with said disease requires is reduced, or may enable the discontinuation of the subject's existing medication.

Preventing a disease or condition relates to the prophylactic use of the cells herein. In this respect, the cells may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations.

A “therapeutically effective amount” is at least the minimum concentration required to affect a measurable improvement of a particular disease, disorder, or condition. A therapeutically effective amount herein may vary according to factors such as the disease state, age, sex, and weight of the patient, and the ability of the chimeric receptors to elicit a desired response in the individual. A therapeutically effective amount is also one in which any toxic or detrimental effects of the cell, cell population or pharmaceutic compositions are outweighed by the therapeutically beneficial effects.

The terms “subject”, “patient” and “individual” are used interchangeably herein and refer to a mammal, preferably a human. In particular, the terms subject, patient and individual refer to a human having a disease or disorder as defined herein in need of treatment.

In some embodiments of the invention, the patient may be subjected to other treatments prior to, contemporaneously with, or after the treatments of the present invention. For instance, in some embodiments, the patient may be treated with other procedures for the treatment of symptoms associated with the disease or disorder.

In some embodiments, the cell, cell population or pharmaceutical composition of the invention may be administered in combination with other therapeutic agents for the treatment of symptoms associated with the disease or disorder or other underlying condition.

The medical use of or method herein may involve the steps of:
(i) isolating a cell-containing sample or providing a cell-containing sample;
(ii) introducing a nucleic acid molecule, construct or a vector as defined herein to the cell; and
(iii) administering the cells from (ii) to a subject.

The cell may be a Treg as defined herein. An enriched Treg population may be isolated and/or generated from the cell containing sample prior to, and/or after, step (ii) of the method. For example, isolation and/or generation may be performed prior to and/or after step (ii) to isolate and/or generate an enriched Treg sample. Enrichment may be performed after step (ii) to enrich for cells and/or Tregs comprising the CAR, the polynucleotide, and/or the vector as described herein.

Suitably, the cell may be autologous. Suitably, the cell may be allogenic.

Suitably, the cell (e.g., the engineered Treg) may be administered in combination with one or more other therapeutic agents, such as lympho-depletive agents (e.g., as discussed above). The engineered cell, e.g., Treg, may be administered simultaneously with or sequentially with (i.e., prior to or after) the one or more other therapeutic agents.

Cells, e.g., Tregs, may be activated and/or expanded prior to, or after, the introduction of a nucleic acid molecule as described herein, for example by treatment with an anti-CD3 monoclonal antibody or both anti-CD3 and anti-CD28 monoclonal antibodies. Expansion protocols are discussed above.

The cell, e.g., Tregs, may be washed after each step of the method, in particular after expansion.

The population of engineered cells, e.g., Treg cells may be further enriched by any method known to those of skill in the art, for example by FACS or magnetic bead sorting.

The steps of the method of production may be performed in a closed and sterile cell culture system.

The invention may also provide a method for increasing the stability and/or suppressive function of a cell comprising the step of introducing a nucleic acid molecule, an expression construct or vector as provided herein into the cell. An increase in suppressive function can be measured as discussed above, for example by co-culturing activated antigen-specific Tconv cells with cells of the invention, and for example measuring the levels the cytokines produced by the Tconv cells. An increase in suppressive function may be an increase of at least 10, 20, 30, 40, 50, 60, 70, 80, or 90% as compared to a non-engineered Treg.

An increase in stability of a cell, e.g., a Treg as defined herein, refers to an increase in the persistence or survival of those cells or to an increase in the proportion of cells retaining a Treg phenotype over a time period (e.g., to cells retaining Treg markers such as FOXP3 and Helios) as compared to a non-engineered Treg.

An increase in stability may be an increase in stability of at least 10, 20, 30, 40, 50, 60, 70, 80 or 90%, and may be measured by techniques known in the art, e.g., staining of Treg cell markers within a population of cells, and analysis by FACS.

The invention also provides use of a CAR-Treg for inducing an anti-inflammatory microglial phenotype. An “anti-inflammatory microglial phenotype” may alternatively be referred to as an M2 microglial phenotype. In the literature, microglia are classically known to have three phenotypes: M0=steady state (or resting) microglial cells, M1=activated microglial cells that release pro-inflammatory molecules such as Reactive Oxygen Species (ROS) and Interleukin (IL) 6 and 8 and are associated with neurotoxicity, and M2=anti-inflammatory microglial cells that promote tissue remodelling and repair by releasing high levels of IL-4 and IL-10. Microglia can shift from an M1 to an M2 phenotype in particular conditions and contribute to neuroprotection. As shown herein, Treg cells can promote this shift to an M2 (or anti-inflammatory) phenotype. Therefore, alternatively viewed, the invention provides use of a CAR-Treg for changing the phenotype of a microglial cell from M1 to M2. Furthermore, the invention provides use of a CAR-Treg for increasing the number of microglial cells expressing the anti-inflammatory marker arginase-1 (ARG1). As an anti-inflammatory marker, expression of ARG1 indicates that microglia have an M2 phenotype.

The microglial cell or cell population may be from any species, but preferably it is a human or mouse microglial cell or cell population.

The CAR may be the CAR of the present invention, i.e., it may comprise an antigen recognition domain that specifically binds to TREM2 (e.g., to human TREM2), and it may have any of the features of the CAR as disclosed herein. This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### Examples

### Materials and Methods

### Cloning

CAR constructs were designed in house, and whole sequences were codon optimised for expression in human or murine cells and manufactured. Constructs were cloned into the retroviral backbone pMP71 backbone and D5a high efficiency bacteria were transformed with plasmid and grown with the selection agent ampicillin. Constructs 3, 7, 8 and 10 represent constructs of the present invention as claimed herein (i.e., anti-TREM2 CARs). The constructs as used herein also contain GFP to assess transduction efficiency (in addition to using the CAR for this purpose).

### Transfection and Viral Particle Production

### Retroviral Production:

GP2-293 cells were transfected with relevant plasmid DNA and envelope plasmid. 24 h after transfection media was replaced and on day 3 supernatant containing retrovirus was harvested from the GP2-293 cells and filtered. On day 4, the viral supernatant was concentrated.

### Transduction of Jurkat NFAT Cells

Jurkat NFAT cells were transduced using the retroviral vector produced as described above (i.e., with viral vectors encoding constructs 3, 7, 8 or 10 or with a negative control construct that does not comprise an anti-TREM2 scFv) and cultured for 24 hrs. On the next day, the Jurkat NFAT cells were harvested and resuspended in fresh media.

### Transduction of Murine Tregs

Murine Tregs were transduced using the retroviral vector produced as described above (i.e., with viral vectors encoding constructs 3, 7, 8 or 10 or with a negative control construct that does not comprise an anti-TREM2 scFv). 24 h or 48 h after transduction, murine Tregs were fed with fresh media. Transduced murine Tregs were sustained in culture by adding fresh media every other day.

### Flow Cytometry Staining to Determine CAR Expression in Jurkat NFAT Cells

On day 4 after transduction, Jurkat NFAT cells were stained for surface expression of the anti-TREM2 CARs using Biotinylated Human TREM2 Protein (TR2-H82E7 ACRObiosystems) and Streptavidin-APC as a secondary antibody. On day 7, CAR expressing cells were selected using Streptavidin magnetic beads (Streptavidin MicroBeads, 130-048-101, Miltenyi), columns (Miltenyi) and Biotinylated Human TREM2 Protein (TR2-H82E7 ACRObiosystems) and then used for subsequent activation assays.

### Flow Cytometry Staining to Determine CAR Expression in Murine Tregs

On day 5 after transduction, murine Tregs were stained for surface expression of the anti-TREM2 CARs using Biotinylated Human TREM2 Protein (TR2-H82E7 ACRObiosystems) and Streptavidin-APC as a secondary antibody. GFP was additionally detected as a marker of CAR expression. The cells were analysed by a flow-cytometer.

CAR-Transduced Jurkat NFAT Cell Activation Assay with TREM2 Proteins

On day 0, plates were left uncoated (non-stimulated condition) or were coated with a purified anti-human CD3 antibody (317301, Biolegend, clone: OKT3) as a positive control (50 ug/ml) or with various TREM2 proteins, as follows:
Biotinylated Human TREM2 Protein,His,Avitag™ (TR2-H82E7, ACRObiosystems) (abbr: huTREM2,Biotin,His,Avitag).
TREM2, Fc-fusion (IgG1), Avi-Tag (Mouse), Biotin (79459, BPSBioscience) (abbr: muTREM2, Fc-fusion).
Recombinant Mouse TREM2 Fc Chimera Protein, CF (1729-T2-050, R&Dsystems) (abbr: muTREM2, Fc Chimera CF).

On day 1, protein was removed and wells were washed, prior to seeding the anti-TREM2-CAR expressing Jurkat NFAT cells or non-transduced cells into each well. On day 2, cells were transferred to white 96 well plates for luminescence assessment. Substrate (ONE-Glo™ Luciferase Assay System, E6110, Promega) was added and samples were read using a plate reader.

CAR-Transduced Jurkat NFAT Cell Activation Assay with TREM2 Positive Cell Lines

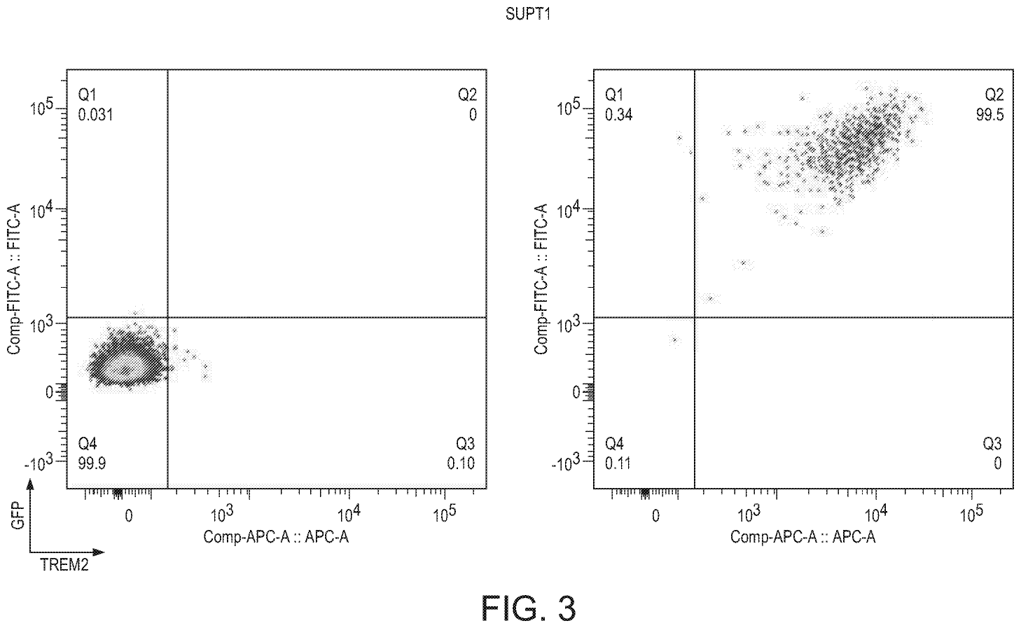

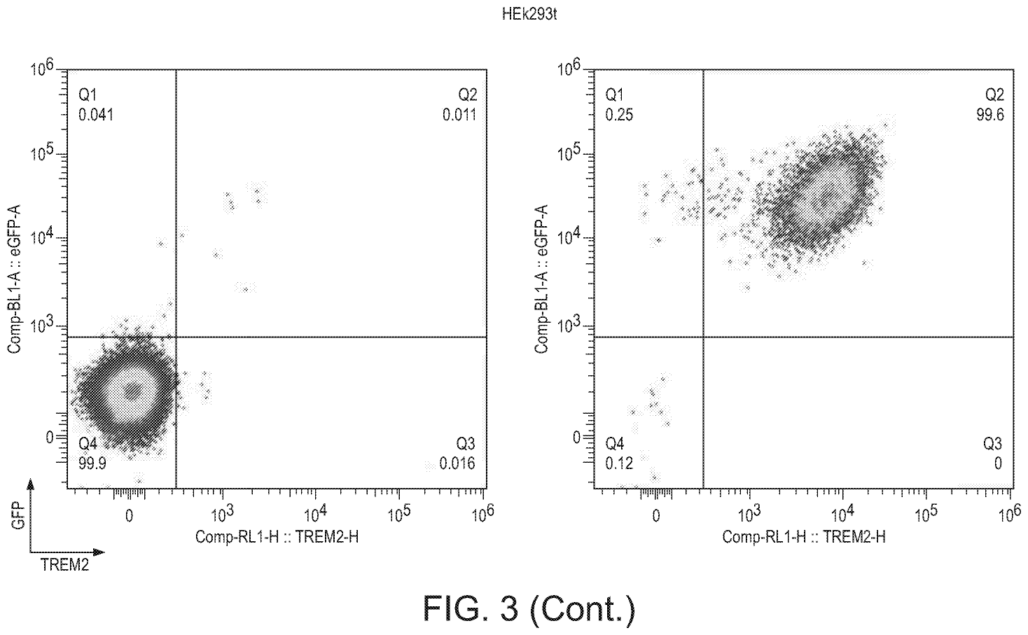

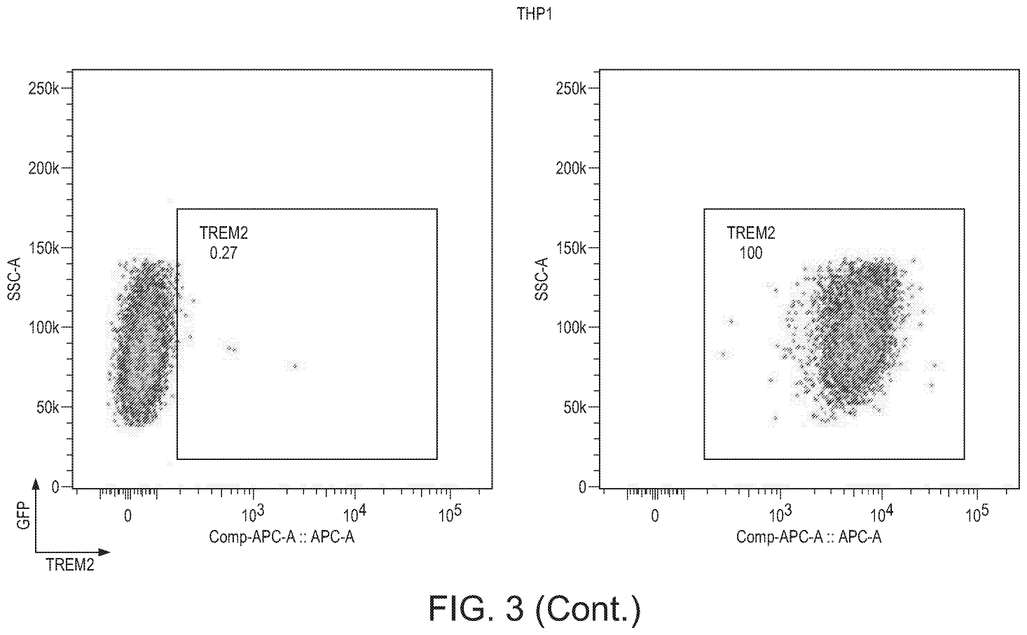

Two target cell lines, SUPT1 and HEK293t, were transduced with TREM2 to over-express the TREM2 protein (see FIG. 3). ‘SUPT1 Mock’ and ‘HEK293t Mock’ cells (i.e., non-transduced cells) were used as a control. As a fourth target cell line the naturally TREM2 expressing cell line THP-1 was used.

On day 1, the transduced and non-transduced target cell lines were seeded in a 1:1 ratio with the anti-TREM2-CAR expressing Jurkat NFAT cells. HEK293t cells were added to plates 2-4 hours prior to addition of the Jurkat cells to allow them to adhere to the plates, whereas SUPT1 and THP1 cells were added at the same time as the CAR expressing Jurkat cells. Jurkat cells were also seeded on their own as a non-stimulated control. On day 2, cells were transferred to white 96 well plates for luminescence. Substrate (ONE-Glo™ Luciferase Assay System, E6110, Promega) was added and samples were read using a plate reader.

CAR-Transduced Murine Treg Cell Activation Assay with TREM2 Proteins and TREM2-Positive Cell Lines

On day 5 post transduction, murine Tregs were resuspended in fresh media (day 0). On the same day, murine and human TREM2 proteins were immobilised in a 96-well non-tissue culture plate and incubated overnight at 4° C. Additionally, wild-type (WT) HEK293 or HEK293 cells expressing either murine or human TREM2 were plated in a tissue-culture treated 96-well plate and incubated overnight in a CO₂ incubator at 37° C. On the following morning (day 1), the wells with immobilised TREM2 proteins were washed twice with PBS. Additionally, THP-1 cells were seeded in a tissue-culture treated 96-well plate. Transduced or mock-transduced Tregs were seeded in each well containing either the proteins, the HEK293 cells or THP-1 cells. Tregs activated with aCD3/aCD28 beads were used a positive control while Tregs cultured with media alone were used as negative controls. 24 h post assay set-up, murine Tregs were analysed for the expression of activation markers e.g. CD137, CD44 and CD69 by flow-cytometry.

### Flow Cytometry to Determine Expression of TREM2 on Cell Lines

Surface expression of TREM2 was investigated in SUPT1 WT, SUPT1 transduced to express human TREM2, HEK293 WT and HEK293 transduced to express human or mouse TREM2 and in THP-1 cells. The cells were Fc-blocked and subsequently stained with an anti-human/mouseTREM2 APC-conjugated antibody. Samples were analysed by a flow-cytometer. Non-transduced cells (for SUPT1 or HEK293 cells) or FMO controls (for THP-1 cells) were used as negative controls.

### In Vivo Trafficking/Activation of TREM2 CAR Transduced CD45.1 Murine T Effector Cells in a SOD1 Mouse Model

CD4 T effector cells (Teffs) from the spleens and lymph nodes of CD45.1 mice were isolated and either non-transduced (mock), transduced with a CAR construct in accordance with the present invention (i.e., an anti-TREM2 CAR construct which expresses GFP, namely construct 8) or transduced with a GFP control. Cells were then frozen down.

On day 0, cells were thawed and 1×10⁶ Teff cells/mouse were injected by Intracisterna magna (ICM) injection into SOD1 mice and age matched wild-type (WT) BL/6 mice (n=9 for each group injected with TREM2 and n=6 for each group injected with GFP transduced controls). 1×10⁶ non-transduced Teff cells/mouse were injected into SOD1 and WT mice (n=3 for each group) as an additional control.

On day 6 post-transfer, all mice were sacrificed, and CNS tissue harvested. Tissue was processed into a single cell suspension and prepared for flow cytometry analysis by staining with CD45.1, CD4 and CD25 antibodies (CD25 is a marker of T cell activation).

### In Vivo Trafficking of TREM2 CAR Transduced CD45.1 Murine T Effector Cells in an EAE Mouse Model and Clinical Progression of EAE

CD4 T effector cells (Teffs) from the spleens and lymph nodes of CD45.1 mice were isolated and transduced with a GFP control or CAR construct in accordance with the present invention (i.e., an anti-TREM2 CAR construct which expresses GFP, namely construct 8). Cells were then frozen down.

C57BL/6J male mice at 9 weeks of age were immunized to induce EAE. The CAR transduced Teffs were administered intravenously at 5×10⁶ cells per mouse at D11 post induction of EAE and mice were sacrificed at D16. Spinal cord tissue was harvested and processed into a single cell suspension and prepared for flow cytometry analysis by staining with CD45.1 and CD4 antibodies.

Clinical scores were recorded daily as described in the table below (Table 2).

TABLE 2
Evaluation grid of EAE clinical score

| Clinical Score | Disease evolution |
|---|---|
| 0 | No clinical signs |
| 0.5 | Partial tail paralysis |
| 1 | Tail paralysis or waddling gait |
| 1.5 | Partial tail paralysis and waddling gait |
| 2 | Tail paralysis and waddling gait |
| 2.5 | Partial limb paralysis |
| 3 | Paralysis of one limb |
| 3.5 | Paralysis of one limb and partial paralysis |
| | of another |
| 4 | Complete hind-limb paralysis |
| 4.5 | Complete hind-limb paralysis and front |
| | limb weakness |
| 5 | Death |

Co-Culture of Mouse Tregs with BV2 Murine Microglia Cells

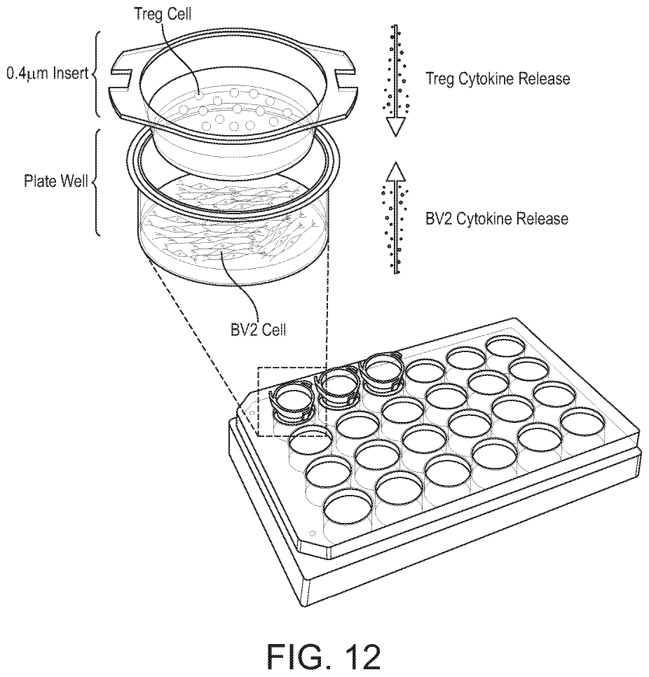

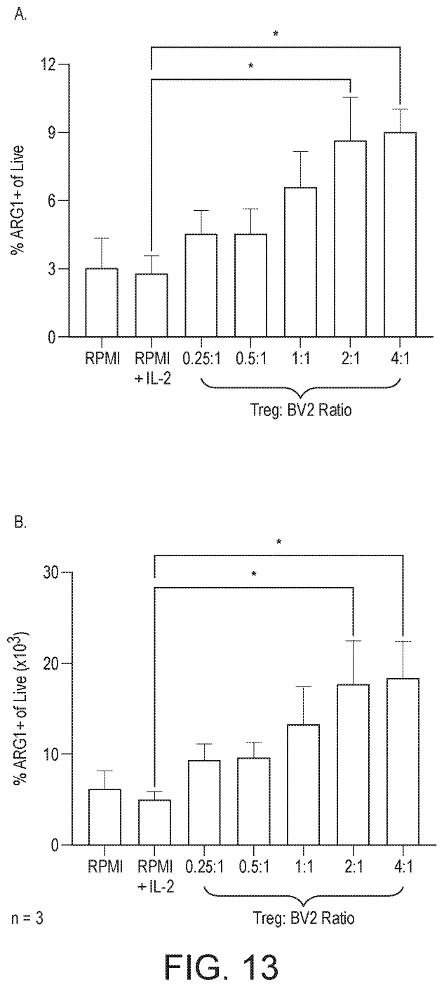

BV2 murine microglial cell lines were added at the bottom of a 24 well plate and rested for 2 hours. Murine Treg cells in a permeable 0.4 μm insert were added to the 24 well plate, so that the cells were not in direct contact, but so that soluble factors could diffuse freely from the Tregs to the BV2 cells and vice versa (see the diagram in FIG. 12). Upon addition of the inserts to the 24 well plate, Tregs were stimulated with anti-CD3/anti-CD28 beads and IL-2 for 24 h. Inserts comprising only Treg medium (RPMI) (but no Tregs) or Treg medium and IL-2 (but no Tregs) were added to the 24 well plates as a control. After 24 h, the percentage of live BV2 cells expressing the anti-inflammatory microglia marker ARG1 was assessed by flow cytometry, as was the number of live BV2 cells expressing ARG1. The results are shown in FIGS. 13a and 13b, respectively.

### Results

### Experiment 1

### Flow Cytometry Staining to Determine CAR Expression in Jurkat NFAT Cells

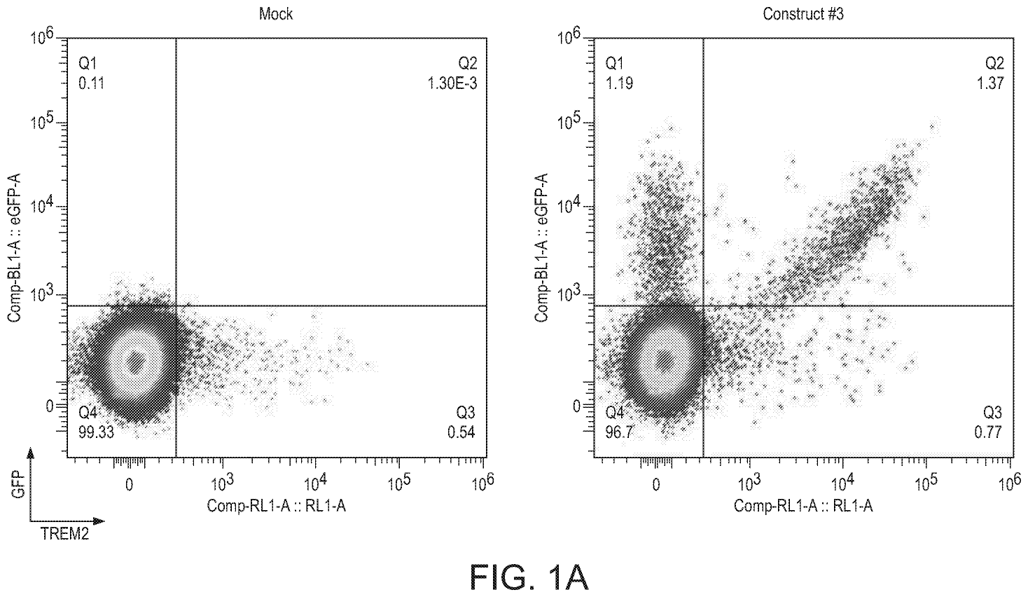

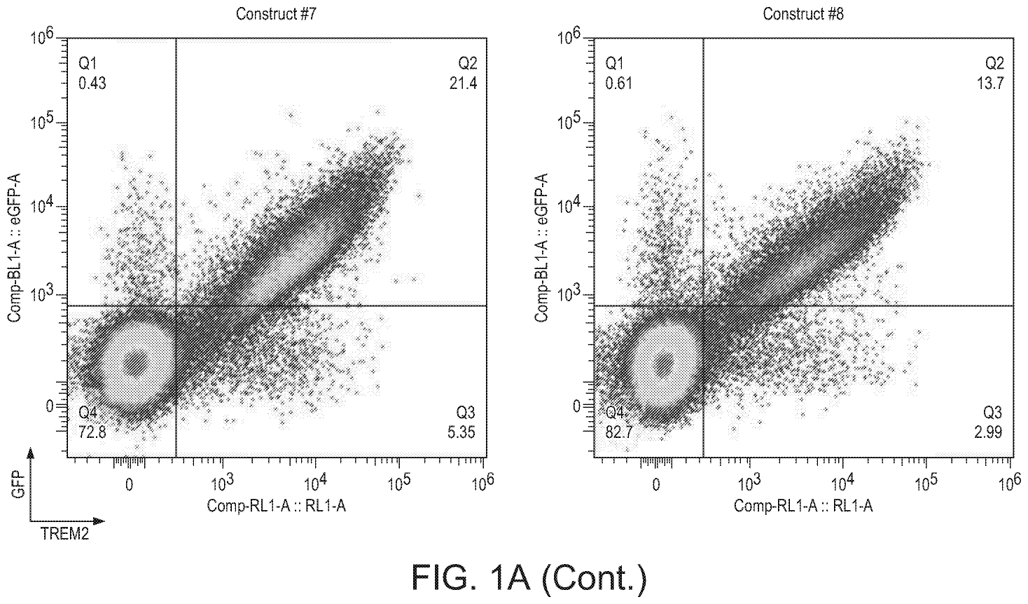

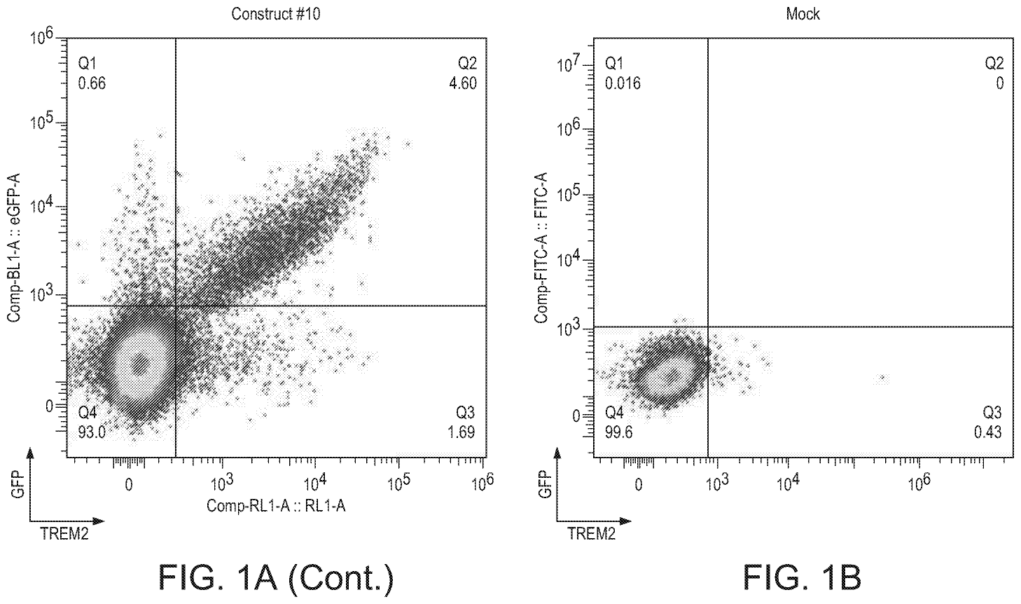

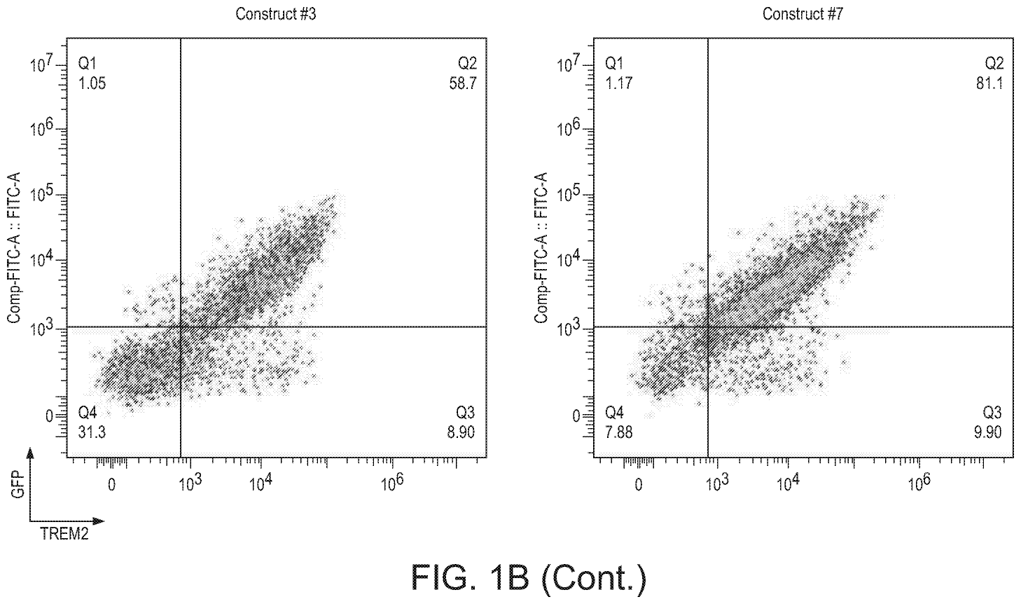

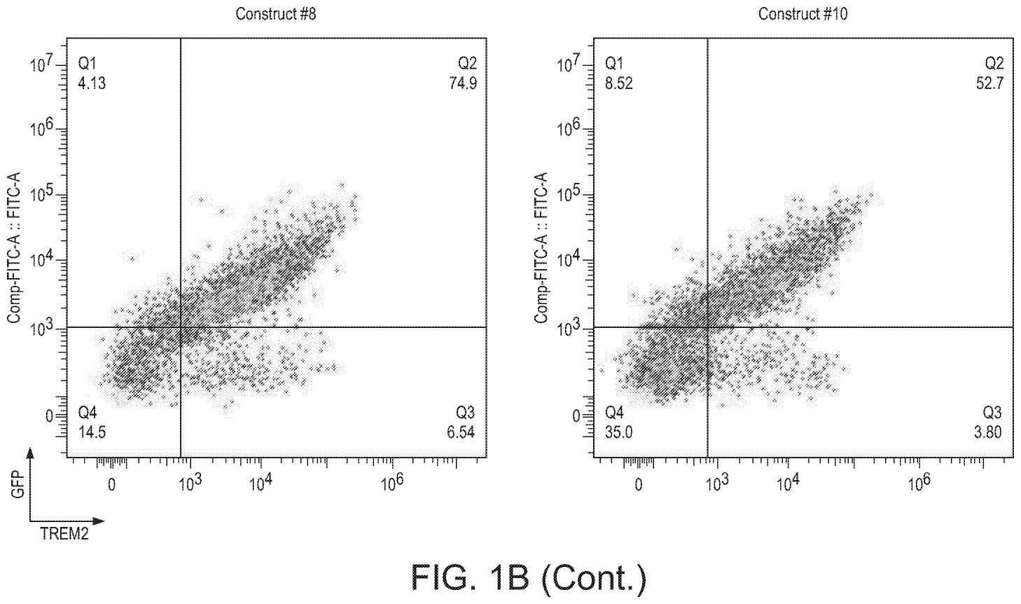

Non-transduced Jurkat NFAT cells and CAR-transduced (i.e., CAR-specific) Jurkat NFAT cells were assessed by flow cytometry for expression of the anti-TREM2 CAR constructs of the present invention (i.e., constructs 3, 7, 8 or 10) using a Biotinylated Human TREM2 Protein and a Streptavidin APC antibody. FIG. 1a shows that the non-transduced cells had very little staining for GFP and TREM2 as expected, whereas the transduced cells expressed both GFP and TREM2 showing that constructs 3, 7, 8 and 10 were all effectively expressed in these cells. FIG. 1b shows high expression of constructs 3, 7, 8 and 10 after sorting (i.e., enriching) for Jurkat NFAT cells expressing these constructs.

### Experiment 2

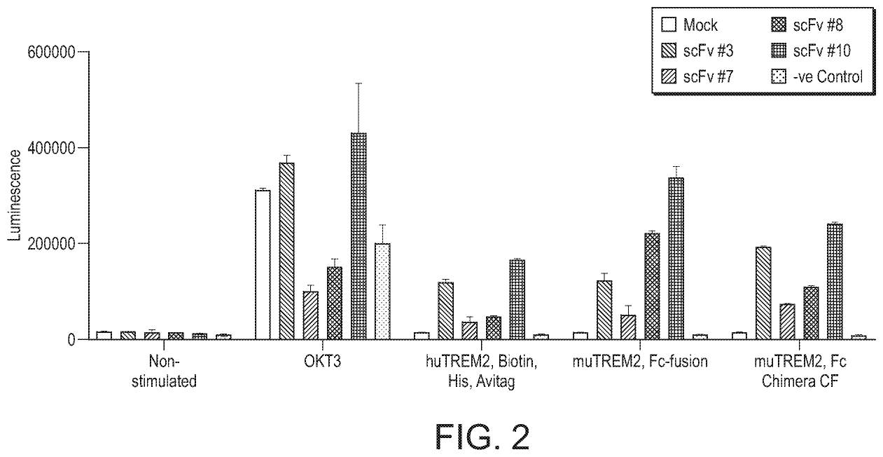

CAR-transduced Jurkat NFAT cell activation assay with TREM2 proteins Non-transduced Jurkat NFAT cells, CAR-transduced (i.e., CAR-specific) Jurkat NFAT cells and Jurkat NFAT cells transduced with a negative control construct were either unstimulated or activated with an anti-CD3 antibody (OKT3) as a positive control or with various human or murine TREM2 proteins (huTREM2, Biotin,His,Avitag; muTREM2,Fc-Fusion; muTREM2, Fc Chimera CF). FIG. 2 shows that Jurkat NFAT cells transduced with constructs 3, 7, 8 or 10 were activated by the positive control and by both human and mouse TREM2 proteins, showing that these constructs are cross-reactive.

### Experiment 3

CAR-Transduced Jurkat NFAT Cell Activation Assay with Cell Lines Over-Expressing TREM2

SUPT1 and HEK293t target cell lines were engineered to over-express TREM2. THP-1 cells express TREM2 naturally. FIG. 3 shows that all three cell lines had high expression of TREM2 when assessed by flow cytometry.

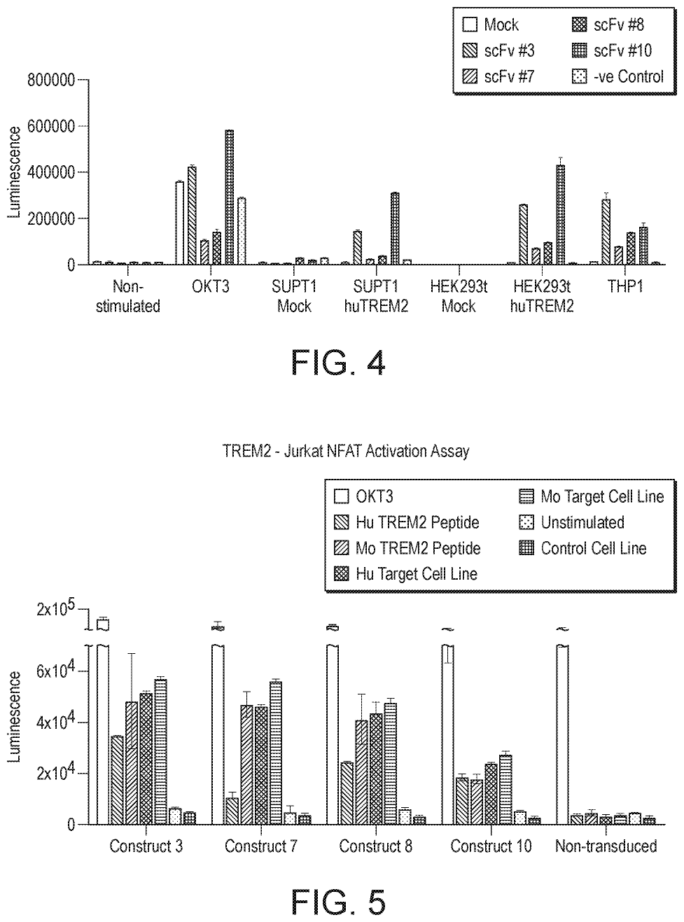

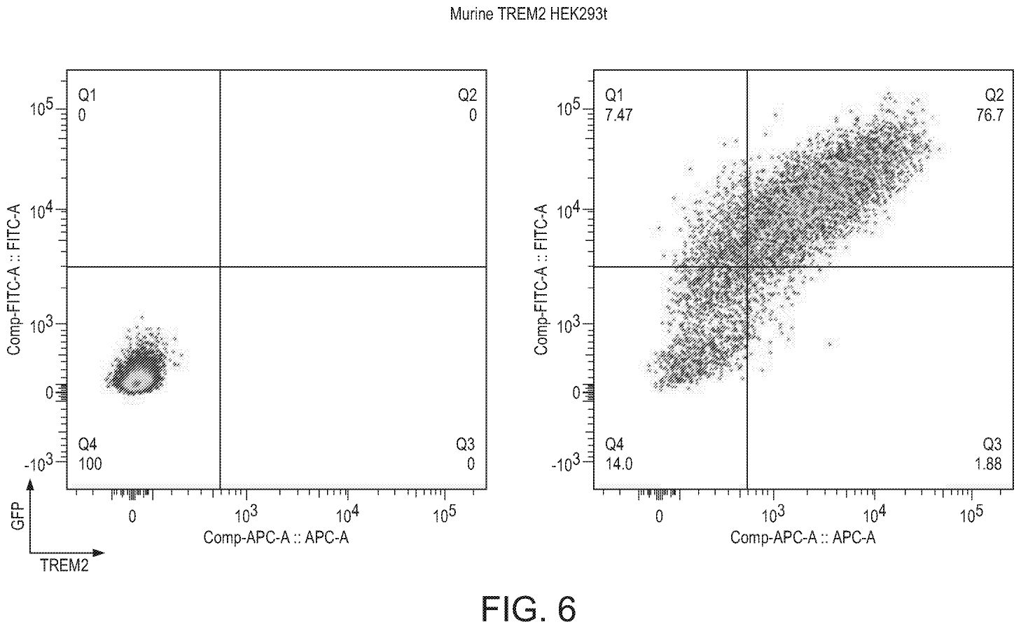

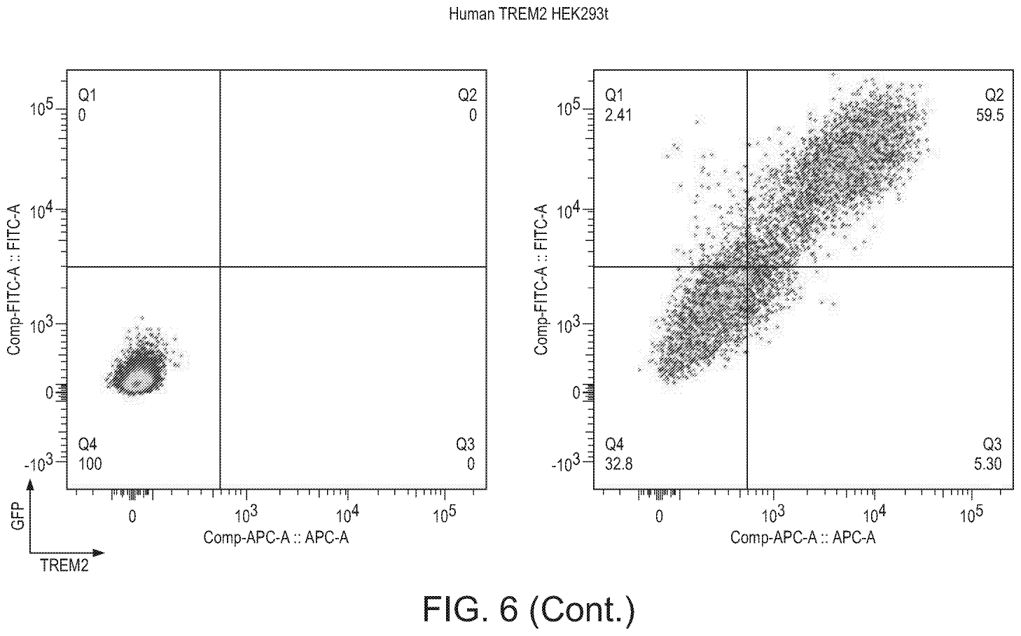

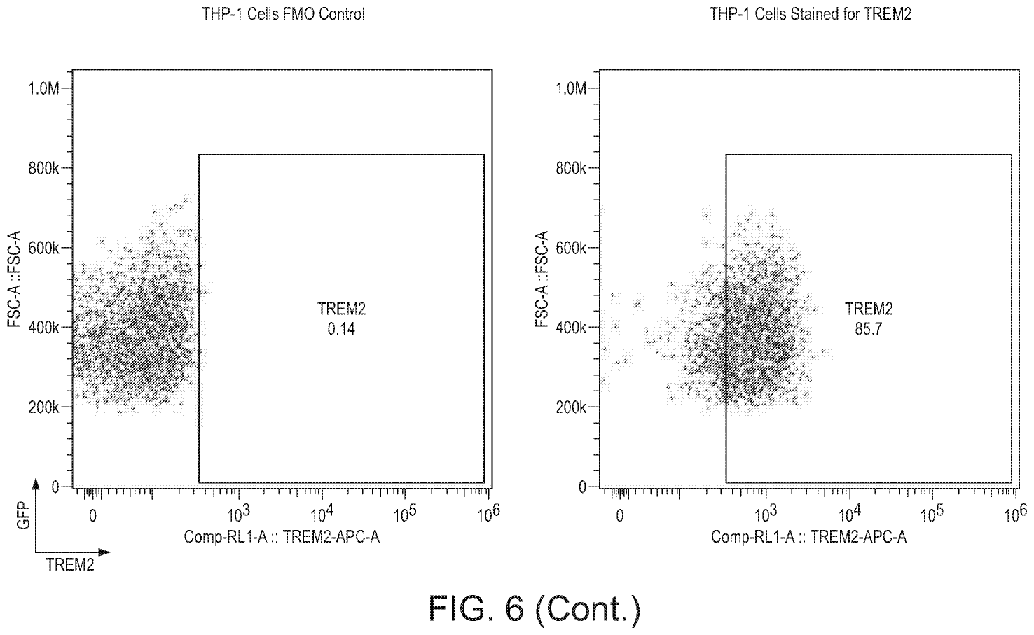

Non-transduced Jurkat NFAT cells, CAR-transduced (i.e., CAR-specific) Jurkat NFAT cells and Jurkat NFAT cells transduced with a negative control construct were either unstimulated, contacted with the target cell lines over-expressing TREM2 or contacted with mock target cell lines (i.e., non-transduced target cell lines). As can be seen in FIG. 4, the Jurkat NFAT cells transduced with constructs 3, 7, 8 or 10 were activated by each of the target cell lines over-expressing TREM2.

### Experiment 4

CAR-Transduced Jurkat NFAT Cell Activation Assay with TREM2 Proteins and with Cell Lines Over-Expressing TREM2

Non-transduced Jurkat NFAT cells or CAR-transduced (i.e., CAR-specific) Jurkat NFAT cells were either unstimulated or activated with an anti-CD3 antibody (OKT3) as a positive control or with Biotinylated human TREM2 protein with a streptavidin tag, murine TREM2 protein with a His-tag, human HEK293T cells over-expressing TREM2 or with mouse HEK293T cells over-expressing TREM2 or were contacted with non-transduced HEK293T cells as a negative control. FIG. 5 shows that Jurkat NFAT cells transduced with constructs 3, 7, 8 or 10 were activated by the positive control and with human and mouse TREM2 proteins and human and mouse cell lines over-expressing TREM2, showing that these constructs can be activated and are cross-reactive.

### Experiment 5

### Flow Cytometry Staining to Determine CAR Expression in Murine Treg Cells

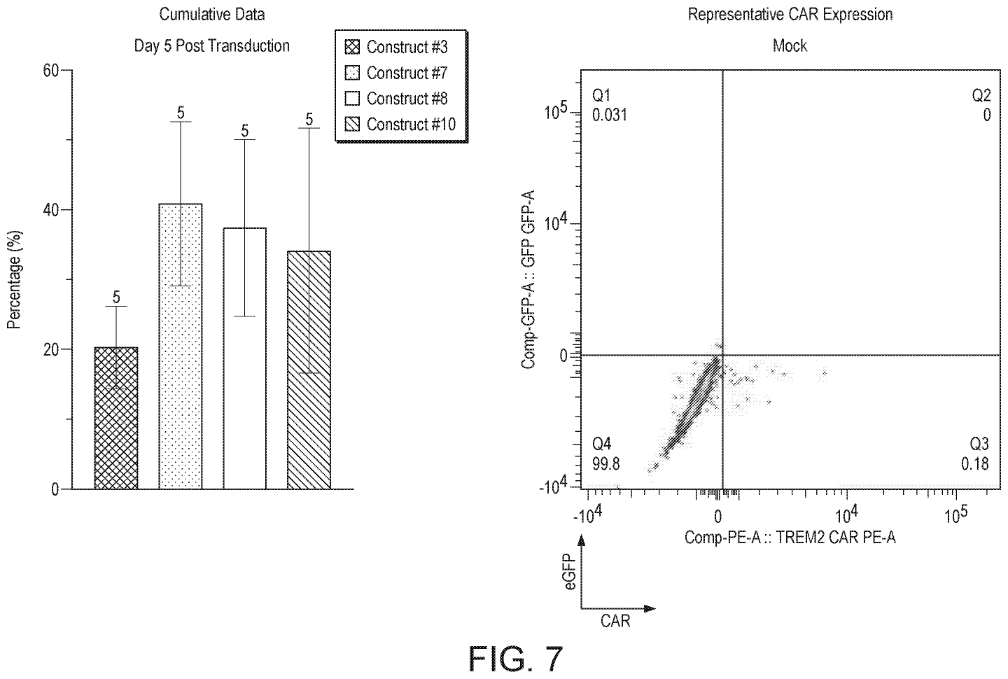

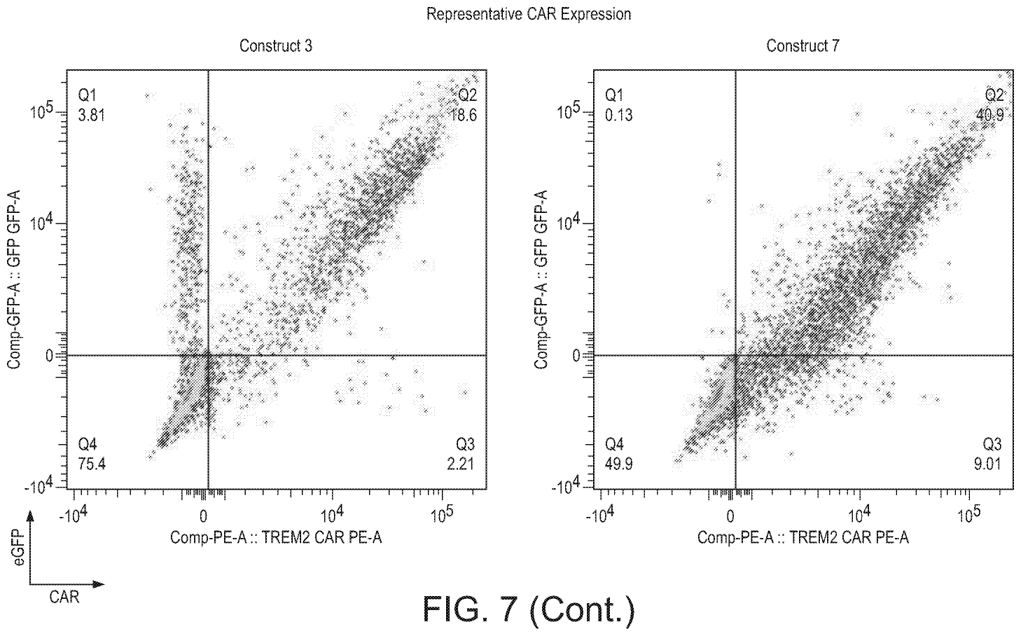

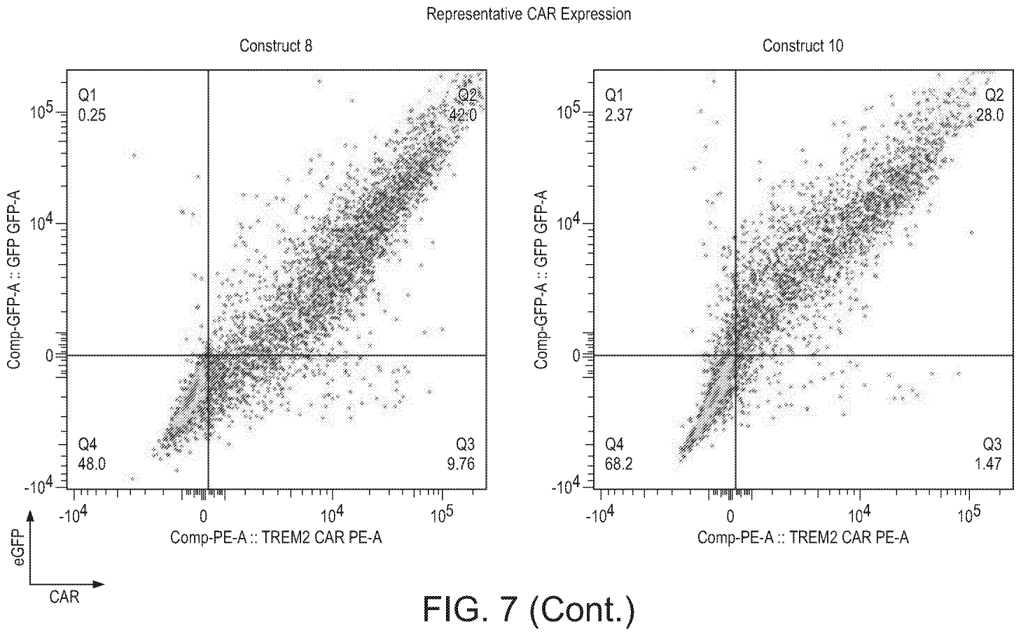

Non-transduced murine Treg cells (“mock”) and CAR-transduced (i.e., CAR-specific) murine Treg cells were assessed by flow cytometry for expression of the anti-TREM2 CAR constructs of the present invention (i.e., constructs 3, 7, 8 or 10) using a Biotinylated Human TREM2 Protein and a Streptavidin APC antibody. FIG. 7 shows that the non-transduced cells (“mock”) had very little staining for GFP and TREM2 as expected, whereas the transduced cells expressed both GFP and TREM2 showing that constructs 3, 7, 8 and 10 were all effectively expressed in these cells.

### Experiment 6

CAR-Transduced Murine Treg Cell Activation Assay with TREM2 Proteins and TREM2-Positive Cell Lines

Non-transduced murine Treg cells (“mock”) or CAR-transduced (i.e., CAR-specific) murine Treg cells were either unstimulated or activated with anti-CD3/anti-CD28 beads as a positive control or with Biotinylated human TREM2 protein with a streptavidin tag, murine TREM2 protein with a His-tag, human HEK293 cells over-expressing TREM2, mouse HEK293 cells over-expressing TREM2, or were contacted with non-transduced HEK293 cells as a negative control (“HEK293 WT”). 24 h post assay set-up, murine Tregs were analysed for the expression of activation markers e.g. CD137, CD44 and CD69 by flow-cytometry.

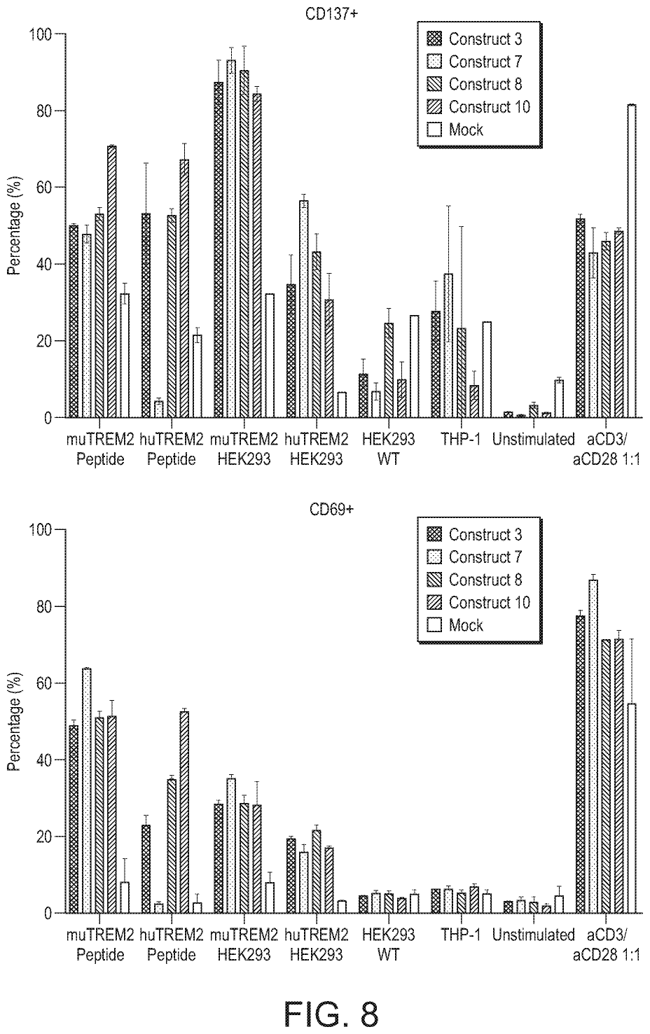

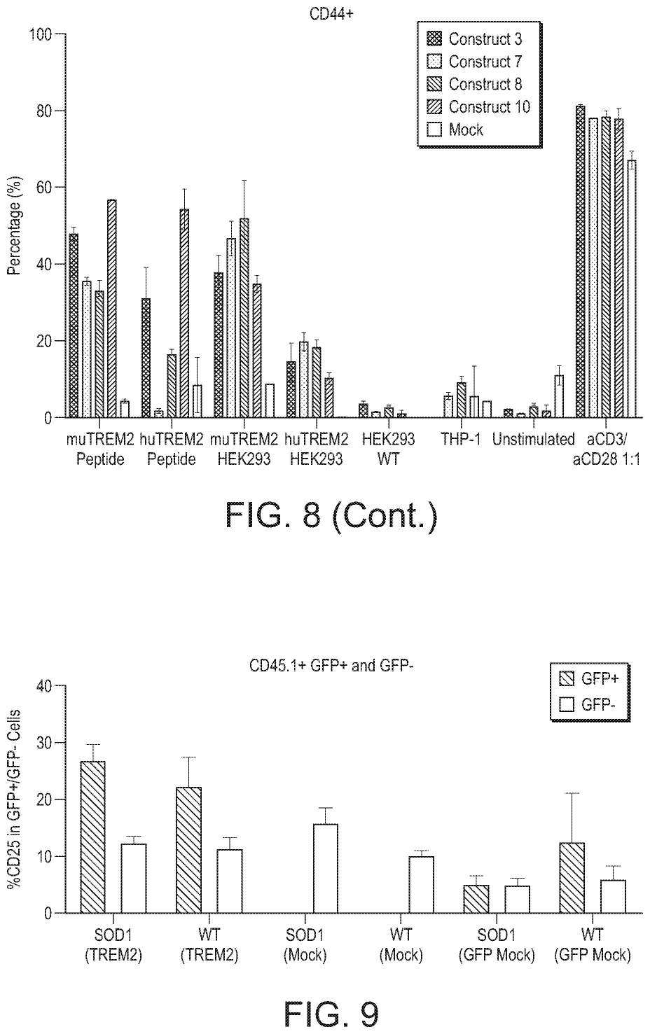

FIG. 8 shows that murine Treg cells transduced with constructs 3, 7, 8 or 10 were activated by the positive control and by human TREM2 protein (with the exception of construct 7) and mouse TREM2 protein, by human and mouse cell lines over-expressing TREM2, and to some extent by THP-1 cells, showing that these constructs can be activated and are cross-reactive.

### Experiment 7

### In Vivo Trafficking and Activation of TREM2 CAR Transduced CD45.1 Murine T Effector Cells in a SOD1 Mouse Model

As mentioned above, CD4 T effector cells (Teffs) from the spleens and lymph nodes of CD45.1 mice were isolated and either non-transduced (mock), transduced with a CAR construct in accordance with the present invention (i.e., an anti-TREM2 CAR construct which expresses GFP, namely construct 8) or transduced with a GFP control and then injected by ICM into SOD1 or WT mice.

On day 6 post-transfer, all mice were sacrificed, and CNS tissue harvested. Tissue was processed into a single cell suspension and prepared for flow cytometry analysis by staining with CD45.1, CD4 and CD25 antibodies.

FIG. 9 shows that the CD45.1+ GFP+ (i.e. injected, CAR-transduced) cells accumulated in the CNS at day 6 and were specifically activated by their target (i.e. TREM2) in the CNS (as indicated by the amount of CD25 expression on the Y axis).

### Experiment 8

### In Vivo Trafficking of TREM2 CAR Transduced CD45.1 Murine T Effector Cells in an EAE Mouse Model and Clinical Progression of EAE

As mentioned above, CD4 T effector cells (Teffs) from the spleens and lymph nodes of CD45.1 mice were isolated and transduced with a GFP control or CAR construct in accordance with the present invention (i.e., an anti-TREM2 CAR construct which expresses GFP, namely construct 8) and then injected intravenously into EAE (“immunized”) or WT (“non-immunized”) mice.

On day 16 post-transfer, all mice were sacrificed, and spinal cord tissue harvested. Tissue was processed into a single cell suspension and prepared for flow cytometry analysis by staining with CD45.1 and CD4 antibodies. Clinical scores were also recorded daily as shown in Table 2 above.

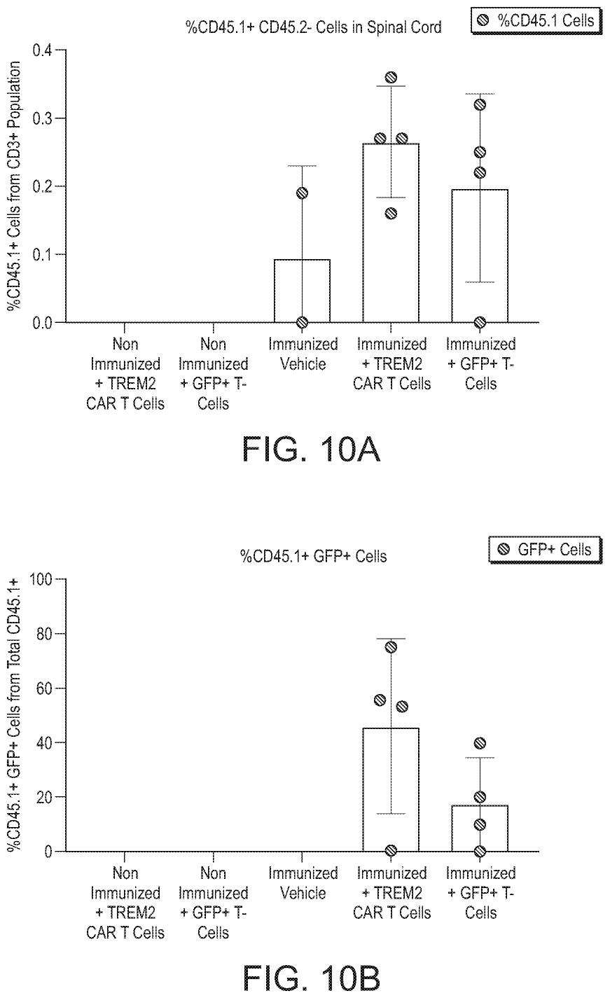

FIG. 10, left-hand side, shows that the CD45.1+ cells (i.e., the injected cells) were present in the spinal cord of the EAE mice, but not in the spinal cord of the WT mice, at day 16. FIG. 10, right-hand side, shows the percentage of those CD45.1+ cells present in the spinal cord that were GFP+ (i.e. that had been successfully transduced with the TREM2 CAR).

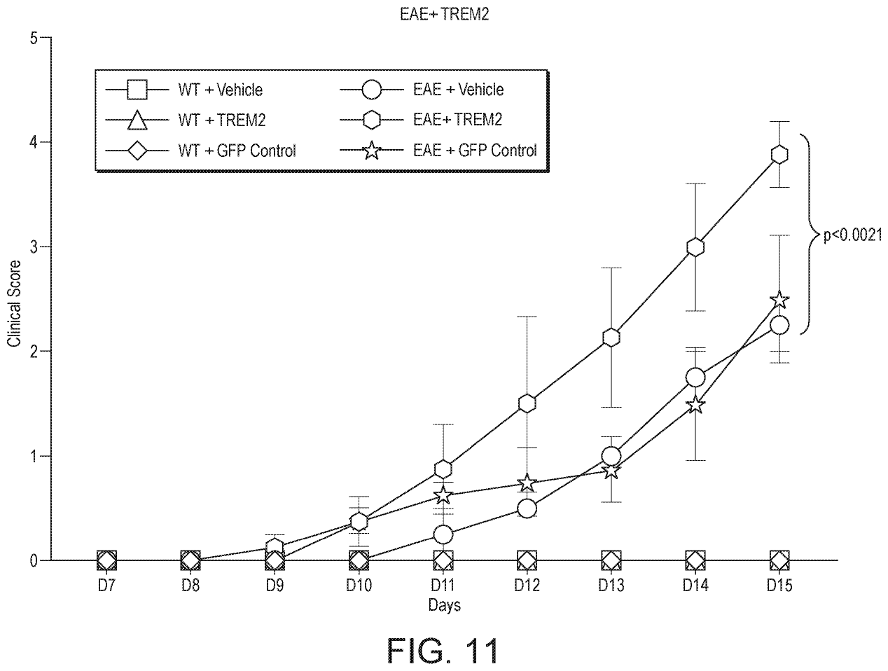

FIG. 11 shows that in the mice that had been immunized to induce EAE, those that received murine Teffs transduced with the TREM2 CAR had significantly enhanced disease progression as compared to those that had received the vehicle or murine Teffs transduced with the GFP control. This indicates that the TREM2 CAR has been activated and is causing the murine Teffs to exert their inflammatory effects, worsening disease. As expected, the WT mice showed no disease progression in any of the conditions.

T effector cells (Teffs) are often used in in vivo studies instead of T regulatory cells (Tregs) as they are easier to produce than Tregs and can be obtained in higher numbers. However, it is important to note that Teffs have the opposite effect to Tregs in that they enhance inflammation, whereas Tregs suppress it. Therefore, it is expected that in the EAE model, murine Tregs transduced with the TREM2 CAR would reduce disease progression rather than enhance it.

### Experiment 9

Co-Culture of Mouse Tregs with BV2 Murine Microglia Cells

As mentioned above, mouse Treg cells and murine microglial cells were cultured together in a co-culture experiment where the cells were not in direct contact, but where soluble factors could diffuse freely from the Tregs to the BV2 cells, and vice versa (see the diagram in FIG. 12). Permeable inserts comprising Treg cells were added to 24 well plates comprising BV2 cells, and the Treg cells were then stimulated with anti-CD3/anti-CD28 beads and IL-2 for 24 h. Inserts comprising only Treg medium (but no Tregs), or Treg medium and IL-2 (but no Tregs) were added to the 24 well plate as a control. After 24 h, the percentage of live BV2 cells expressing the anti-inflammatory microglia marker ARG1 was assessed by flow cytometry, as was the number of live BV2 cells expressing ARG1. Results are polled from n=3 experiments.

As can be seen in FIG. 13, after 24 h of co-culture, the percentage (%) of live BV2 cells (FIG. 13a) and the number (#) of live BV2 cells (FIG. 13b) expressing the anti-inflammatory microglia marker ARG1 increased in the cells that had been co-cultured with Tregs as compared to the cells that had been co-cultured only with Treg medium and IL-2. This increase was significant at a 2:1 and 4:1 Treg:BV2 co-culture ratio, with p values of 0.0259 and 0.0172, respectively (statistics were done using an ordinary one-way ANOVA with Dunnett post-hoc testing against the RPMI+IL-2 control group and all other experimental groups). This shows that mouse Tregs release soluble factors that promote induction of an anti-inflammatory BV2 microglial phenotype, suggesting that Tregs can regulate inflammation in the CNS.

1. A chimeric antigen receptor (CAR) comprising an antigen recognition domain that specifically binds to TREM2.

2. The CAR according to claim 1, wherein the antigen recognition domain binds to human TREM2.

3. The CAR of claim 1 or 2, comprising:
a. an exodomain comprising the antigen recognition domain;
b. a transmembrane domain; and
c. an endodomain comprising an intracellular signalling domain.

4. The CAR according to claim 3, further comprising a hinge domain and/or one or more co-stimulatory domains.

5. The CAR according to claim 4, wherein the hinge domain is selected from the hinge regions of CD28, CD8α, CD4, CD7, CH2CH3, an immunoglobulin, or a part or variant thereof, preferably wherein the CAR comprises a CD8α or CH2CH3 hinge domain.

6. The CAR according to any one of claims 3 to 5, wherein the CAR comprises one or more transmembrane domains selected from the transmembrane domains of CD28, ICOS, CD8α, CD4, CD134 (OX40), CD137 (4-1BB), CD3 zeta, CD45, CD9, CD16, CD22, CD33, CD64, CD80, CD86, CD154, CH2CH3, or a part or variant thereof, preferably wherein the CAR comprises a CD8α or CH2CH3 transmembrane domain.

7. The CAR according to any one of claims 4 to 6, wherein the co-stimulatory domain is selected from the intracellular domains of CD28, ICOS, CD134 (OX40), CD137 (4-11BE), CD27, or TNFRSF25, or a part or variant thereof, preferably wherein the CAR comprises a CD28 co-stimulatory domain.

8. The CAR according to any one of claims 3 to 7, wherein the CAR comprises one or more intracellular signalling domains selected from the group consisting of the CD3 zeta signalling domain or any of its homologs, a CD3 polypeptide, a syk family tyrosine kinase, a src family tyrosine kinase, CD2, CD5 and CD8, or a part of variant thereof, preferably wherein the CAR comprises the CD3 zeta signalling domain.

9. The CAR according to any one of claims 3 to 8, wherein the CAR comprises: a CD8α or CH2CH3 hinge domain; a CD8α or CH2CH3 transmembrane domain; a CD28 co-stimulatory domain; and the CD3 zeta signalling domain, wherein when the hinge domain is CD8α, the transmembrane domain is CD8α and when the hinge domain is CH2CH3, the transmembrane domain is CH2CH3.

10. The CAR according to any one of claims 3 to 9, wherein the CAR comprises a signal peptide and/or a reporter peptide.

11. The CAR according to any one of claims 3 to 10, wherein the endodomain comprises a STAT5 association motif, a JAK1 and/or JAK 2 binding motif and optionally a JAK 3 binding motif, preferably wherein the endodomain of the CAR comprises one or more sequences from an endodomain of an interleukin receptor (IL) receptor.

12. The CAR according to any preceding claim, wherein the antigen recognition domain is an antibody, an antibody fragment, or derived from an antibody.

13. The CAR according to any preceding claim, wherein the antigen recognition domain is a single chain antibody (scFv).

14. The CAR according to any preceding claim wherein the antigen recognition domain comprises:
(i) VH CDR1, 2 and 3 sequences as set forth in SEQ ID NOs: 1, 2 and 3 respectively and VL CDR1, 2 and 3 sequences as set forth in SEQ ID NOs: 4, 5 and 6 respectively,
(ii) VH CDR1, 2 and 3 sequences as set forth in SEQ ID NOs: 7, 8 and 9 respectively and VL CDR1, 2 and 3 sequences as set forth in SEQ ID NOs: 10, 11 and 12 respectively,
(iii) VH CDR1, 2 and 3 sequences as set forth in SEQ ID NOs: 13, 14 and 15 respectively and VL CDR1, 2 and 3 sequences as set forth in SEQ ID NOs: 16, 17 and 18 respectively, or
(iv) VH CDR1, 2 and 3 sequences as set forth in SEQ ID NOs: 19, 20 and 21 respectively and VL CDR1, 2 and 3 sequences as set forth in SEQ ID NOs: 22, 23 and 24 respectively,
wherein one or more of said CDR sequences of (i)-(iv) may optionally comprise 1 to 3 amino acid modifications relative to an aforementioned CDR sequence, particularly wherein one or more of said CDR sequences may optionally be modified by substitution, addition or deletion of 1 to 3 amino acids.

15. The CAR according to claim 14 wherein the antigen recognition domain comprises:
a. a VH domain comprising the sequence as set forth in SEQ ID NO: 25, or a sequence having at least 70% sequence identity thereto, and a VL domain comprising the sequence as set forth in SEQ ID NO: 26, or a sequence having at least 70% identity thereto; or
b. a VH domain comprising the sequence as set forth in SEQ ID NO: 27, or a sequence having at least 70% sequence identity thereto, and a VL domain comprising the sequence as set forth in SEQ ID NO: 28, or a sequence having at least 70% identity thereto; or
c. a VH domain comprising the sequence as set forth in SEQ ID NO: 29, or a sequence having at least 70% sequence identity thereto, and a VL domain comprising the sequence as set forth in SEQ ID NO: 30, or a sequence having at least 70% identity thereto; or
d. a VH domain comprising the sequence as set forth in SEQ ID NO: 31, or a sequence having at least 70% sequence identity thereto, and a VL domain comprising the sequence as set forth in SEQ ID NO: 32, or a sequence having at least 70% identity thereto.

16. The CAR according to claim 14 or 15, wherein the antigen recognition domain comprises:
a. the sequence as set forth in SEQ ID NO: 33 or a sequence having at least 80% identity thereto; or
b. the sequence as set forth in SEQ ID NO: 34 or a sequence having at least 80% identity thereto; or
c. the sequence as set forth in SEQ ID NO: 35 or a sequence having at least 80% identity thereto; or
d. the sequence as set forth in SEQ ID NO: 36 or a sequence having at least 80% identity thereto.

17. A nucleic acid molecule encoding the CAR of any preceding claim.

18. A vector comprising the nucleic acid molecule of claim 17.

19. The vector of claim 18, further comprising a nucleic acid molecule encoding a FOXP3 polypeptide.

20. A cell comprising the CAR of any one of claims 1 to 16, the nucleic acid molecule of claim 17 or the vector of claim 18 or 19.

21. The cell of claim 20, wherein the cell is a production host cell.

22. The cell of claim 20, wherein the cell is an immune cell or a progenitor or precursor thereof, preferably a T cell, or a precursor thereof, or a stem cell.

23. The cell of claim 20 or 22, wherein the cell is a Treg, or a precursor thereof, or an iPSC cell.

24. A cell population comprising a cell of any one of claims 20, 22 or 23.

25. A pharmaceutical composition comprising the cell of any one of claims 20, 22 or 23, the cell population of claim 24 or the vector of claim 18 or 19.

26. The cell of any one of claims 20, 22 or 23, the cell population of claim 24, or the pharmaceutical composition of claim 25 for use in therapy.

27. The cell, cell population, or pharmaceutical composition for use according to claim 26, wherein the therapy is adoptive cell transfer therapy.

28. The cell of any one of claims 20, 22 or 23, cell population of claim 24, or pharmaceutical composition of claim 25, for use in treating a neurological disease, disorder or injury, such as a neurodegenerative disease, or autoimmune or inflammatory disease, or for use in inducing immunosuppression, or for use in promoting tissue repair and/or tissue regeneration.

29. The cell, cell population, or pharmaceutical composition for use according to claim 28, wherein the neurodegenerative disease is amyotrophic lateral sclerosis (ALS), frontotemporal dementia (FTD), progressive supranuclear palsy (PSP), Parkinson's disease, Alzheimer's disease, Huntington's Disease or Multiple Sclerosis, preferably wherein the disease is ALS.

30. A method of treating and/or preventing a neurological disease, disorder or injury, such as a neurodegenerative disease, or autoimmune or inflammatory disease; or inducing immunosuppression, or promoting tissue repair and/or tissue regeneration, wherein the method comprises administering a cell of any one of claims 20, 22 or 23, particularly a Treg cell, a cell population of claim 24, or a pharmaceutical composition of claim 25, particularly comprising a Treg.

31. The method according to claim 30, which comprises the following steps:
a. isolation or provision of a Treg-enriched cell sample from a subject;
b. introduction into the Treg cells of a nucleic acid molecule of claim 17 or a vector of claim 18 or 19; and
c. administering the Treg cells from (ii) to the subject.

32. Use of a cell of any one of claims 20, 22 or 23, a cell population of claim 24, or a pharmaceutical composition of claim 25, in the manufacture of a medicament for treating and/or preventing a neurological disease, disorder or injury, such as a neurodegenerative disease, or autoimmune or inflammatory disease; or for inducing immunosuppression, or for promoting tissue repair and/or tissue regeneration in a subject, particularly wherein the cell is a Treg.

33. A method of making a cell according to any one of claims 20, 22 or 23, which comprises the step of introducing into the cell (e.g., transducing or transfecting a cell with) the nucleic acid molecule of claim 17 or the vector of claim 18 or 19.

34. The method of claim 33, wherein the cell is a Treg cell, and the method comprises isolating or providing a cell-containing sample comprising Tregs, and/or Tregs are enriched and/or generated from the cell-containing sample prior to or after the step of introducing the nucleic acid molecule or vector into the cell.

35. A cell obtainable by the method of claim 33 or 34.

36. Use of a CAR-Treg for inducing an anti-inflammatory microglial phenotype.

37. Use of a CAR-Treg for increasing the number of microglial cells expressing the anti-inflammatory marker arginase-1 (ARG1).

38. The use of claim 36 or 37, wherein the CAR is as claimed in any one of claims 1 to 16.
